(19) United States
(12) Patent Application Publication
(10) Pub. No.: US 2020/0315923 A1
(43) Pub. Date: Oct. 8, 2020

(54) DESINFECTANT BONE AND DENTAL FILLER MATERIALS COMPRISING LIPOSOMES

(71) Applicant: Cementic, Villemoisson-Sur-Orge (FR)

(72) Inventor: Samir Raddi, Villemoisson-Sur-Orge (FR)

(21) Appl. No.: 16/652,950

(22) PCT Filed: Oct. 4, 2018
(86) PCT No.: PCT/FR2018/052449

§ 371 Date: Jun. 23, 2020

(30) Foreign Application Priority Data
Oct. 4, 2017 (FR) 1759283

(51) Int. Cl. A61K 6/69; A61K 6/54; A61K 6/15; C12Q 1/18

(52) U.S. Cl. CPC A61K 6/69; C12Q 1/18; A61K 6/15; A61K 6/54

(57) ABSTRACT

Bone or dental filler materials include liposomes, which give them disinfectant properties. These filler materials are used, in particular, for filling dental pulp or a bone defect.

### CROSS-REFERENCE TO RELATED APPLICATIONS

This application is a national phase entry under 35 U.S.C. § 371 of International Patent Application PCT/FR2018/052449, filed Oct. 4, 2018, designating the United States of America and published as International Patent Publication WO 2019/069029 A1 on Apr. 11, 2019, which claims the benefit under Article 8 of the Patent Cooperation Treaty to French Patent Application Serial No. 1759283, filed Oct. 4, 2017.

### TECHNICAL FIELD

The present disclosure pertains to the field of bone or dental filling materials comprising liposomes conferring them disinfecting power. These filling materials are used, in particular, for the filling of dental pulp or bone defects.

### BACKGROUND

Bone is a relatively resilient tissue that has high regenerative capacity compared to other tissues. Under physiological conditions, bone is able to regenerate itself when it comes to small structural defects. In addition, bone destruction resulting from disease, tumor resection, infection or trauma requires surgery involving the replacement of clinically viable bone, which may be autologous, allogenic, xenogenic or synthetic. Disability due to bone loss is therefore a major concern in both orthopedics and oral/maxillofacial surgery.

In dental surgery, since the development of implantology and periodontology, the dental surgeon is faced with a significant number of clinical cases of bone insufficiency. To compensate for these bone defects, the clinician uses bone filling materials of different origins: xenogenic, allogenic, polymeric or mineral such as ceramics.

Also in the dental field, when an infection of the root canals is diagnosed, the practitioner must disinfect and then fill these canals. The care protocol is carried out in several steps. First, the canals are disinfected and shaped, then endodontic cement is used to fill the canals. After about a week, the canal is unclogged over half its length in order to install a pivot (or post) either immediately or 7 days later. The canals are therefore again partially in contact with the external environment during the unclogging and when subsequently placing the pivot, with a risk of inoculating bacteria for about 15 days after the start of treatment.

Dental filling materials are used, in particular, at the end of an endodontic treatment (or “root canal therapy”); they are intended to seal a cone of gutta-percha or to obstruct the pulp/root canals to allow stability of the dental organ. However, as mentioned above, bacteria (persistent or inoculated) frequently grow under the filling material, causing infections that lead to endodontic treatment failure. The preferred solution is then a long and costly endodontic reprocessing with a low success rate (about 40%) or extraction of the tooth, leading to an implant, which is mutilating, cumbersome and expensive.

To prevent post-fill infections and their consequences, different solutions have been proposed, such as UV irradiation or the addition of antiseptic compounds in cements [12].

As an example, International Patent Application Publication No. WO2006/070376 describes an endodontic cement comprising nanoparticles composed of cationic polymers associated with quaternary ammonium (QPEI). This allowed the elimination of bacteria of the genus Streptococcus in an in vitro contact test. The same type of approach is described in International Patent Application Publication No. WO2015/004450, but for a bone cement and the authors propose to add antibiotic-laden liposomes.

None of the solutions proposed in the art is effective in controlling all types of bacteria responsible for dental infections, especially when they are present in the form of biofilm [13]. A study from 2004 showed the advantage of associating chlorhexidine with an endodontic filling material of the mineral trioxide aggregate (MTA) type with a pH of 11.5 in order to improve the disinfecting power of the cement. However, while the presence of chlorhexidine improves disinfection of E. faecalis bacteria, the effect on biofilms is limited [12,13].

The endodontic filling materials commercially available today generally have at best poor or even non-existent antiseptic properties. For sealing bone cements associated with liposomes described in the art (WO2015/004450), the problem of disinfection is different because the sealing stresses are very different from those associated with a filling material. In addition, PMMA (hydrophobic compound), the cement used in this document is not used as a bone filling material (it is, in this context, toxic).

Therefore, a real need [14] persists for a bone and/or dental filling material with disinfectant properties that is effective against the bacteria responsible for bone or dental infections that occur after filling, especially when they are in the form of a biofilm. In particular, such a filling material must ensure complete and lasting disinfection against bacteria in the form of biofilm and must be non-toxic.

### BRIEF SUMMARY

The present disclosure proposes a new type of filling material for dental use, in particular, endodontic, which allows long-term disinfection of the root canal system, the periapex and the dentin tubuli, thanks to the combination of liposomes with disinfecting power. Such a cement can also be used for bone filling.

### BRIEF DESCRIPTION OF THE DRAWINGS

FIG. 1: Diagram of a container suitable for the method of evaluating the disinfecting power of a dental cement.

FIG. 2: Representation of the size distribution of the liposomes formed.

FIG. 3: Representation of the release kinetics of cements comprising different types of liposomes loaded with rhodamine. (A) Sealapex® type calcium hydroxide cement (B) Apexit® type calcium hydroxide cement. The types of liposomes used are illustrated as follows: DOPE: square; EPC: triangle; DODAP: diamond; control without liposome: round.

FIG. 4: Representation of released percentage of the rhodamine contained in cements according to the type of liposomes at 15 days. Ctl=control without liposome.

FIG. 5: Diagram of a mould used for the preparation of cement posts

FIG. 6: Standard curve of a chlorhexidine diglucuronate solution at 255 nm

FIG. 7: Release of chlorhexidine

FIG. 8: Standard curve for determining optical density/bacterial concentration

FIG. 9: Optical Density Measurement at D1

FIG. 10: Optical Density Measurement at D7

FIG. 11: Optical Density Measurement at D20

FIG. 12: Planktonic MDCT graph

FIG. 13: Agar diffusion test

FIG. 14: Chlorhexidine standard curve

FIG. 15: Graph representing the concentration of chlorhexidine (CHX) released over time by a Bio Root™ type filler cement material comprising DOPE liposomes loaded with a 1% CHX solution. The results are shown both in the form of histograms and logarithmic trend curves. Series 1: 0.5% liposome, series 1: 1% liposome, series 3: 3% liposome, series 4: 10% liposome

FIG. 16: Graph representing the concentration of chlorhexidine (CHX) released over time by a Bio Root™ type filler cement material comprising DOPE liposomes loaded with a 5% CHX solution. The results are shown both in the form of histograms and logarithmic trend curves. Series 1: 0.5% liposome, series 2: 1% liposome, series 3: 3% liposome, series 4: 10% liposome

FIG. 17: Graph representing the concentration of chlorhexidine (CHX) released over time by a Bio Root™ type filler cement material comprising DPPC:SA liposomes loaded with a 1% CHX solution. The results are shown both in the form of histograms and logarithmic trend curves. Series 1: 0.5% liposome, series 2: 1% liposome, series 3: 3% liposome, series 4: 10% liposome

FIG. 18: Graph representing the concentration of chlorhexidine (CHX) released over time by a Bio Root™ type filler cement material comprising DPPC:SA liposomes loaded with a 5% CHX solution. The results are shown both in the form of histograms and logarithmic trend curves. Series 1: 0.5% liposome, series 2: 1% liposome, series 3: 3% liposome, series 4: 10% liposome

FIG. 19: Graph representing the concentration of chlorhexidine (CHX) released over time by a Bio Root™ type filler cement material comprising a solution of CHX alone. The results are shown both in the form of histograms and logarithmic trend curves. A=1% CHX (CHX 1% concentrate); B=1% CHX (CHX 5% concentrate); C=10% CHX (CHX 1% concentrate); D 1.0% CHX (CHX 5% concentrate); CTL: negative control (cement without CHX). BA: Bio Root™+A; BB: Bio Root™+B; BC: Bio Root™+C; BD: Bio Root™+D

FIG. 20: Graph representing the concentration of chlorhexidine (CHX) released over time by a Well Root™ type filler cement material comprising DOPE liposomes loaded with a 1% CHX solution. The results are shown both in the form of histograms and logarithmic trend curves. Series 1: 0.5% liposome, series 2: 1% liposome, series 3: 3% liposome, series 4: 10% liposome.

FIG. 21: Graph representing the concentration of chlorhexidine (CHX) released over time by a Well Root™ type filler cement material comprising DOPE liposomes loaded with a 5% CHX solution. The results are shown both as histograms and as logarithmic trend curves. Series 1: 0.5% liposome, series 2: 1% liposome, series 3: 3% liposome, series 4: 10% liposome

FIG. 22: Graph representing the concentration of chlorhexidine (CHX) released over time by a Well Root™ filler cement material comprising DPPC:SA liposomes loaded with a 1% CHX solution. The results are shown both in the form of histograms and logarithmic trend curves. Series 1: 0.5% liposome, series 2: 1% liposome, series 3: 3% liposome, series 4: 10% liposome

FIG. 23: Graph representing the concentration of chlorhexidine (CHX) released over time by a Well Root™ filler cement material comprising DPPC:SA liposomes loaded using a 5% CHX solution. The results are shown both in the form of histograms and logarithmic trend curves. Series 1: 0.5% liposome, series 2: 1% liposome, series 3: 3% liposome, series 4: 10% liposome

FIG. 24: Graph representing the concentration of chlorhexidine (CHX) released over time by a Well Root™ type filler cement material comprising a 5% solution of CHX. The results are shown both in the form of histograms and logarithmic trend curves. A=1% CHX (1% CHX concentrate); B=1% CHX (5% CHX concentrate); C=10% CHX (1% CHX concentrate); D=10% CHX (5% CHX concentrate); CTL: negative control (cement without CHX). WA: Well Root™+A; WB: Well Root™+B; WC: Well Root™+C; WD: Well Root™+D.

FIG. 25: Curve representing the size of the inhibition zone as a function of chlorhexidine concentration in an agar diffusion experiment.

FIG. 26: Inhibition zone created by the samples A) Bio Root™ cement with 1% liposome solution added, B) Bio Root™ alone. Results after 24 hours of incubation.

FIG. 27: Agar diffusion experiment. Bio Root™+1% CEM CHX 0918-001 after 7 days of incubation in agar before inoculation with E. faecalis.

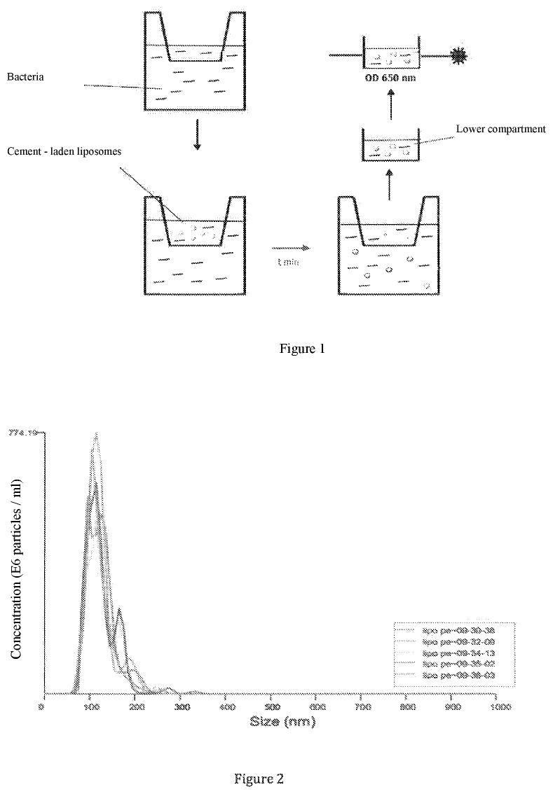

### DETAILED DESCRIPTION

The inventors developed compositions of dental or bone filling materials containing liposomes and demonstrated their ability to release antiseptics for at least 15 days, the time usually elapsing between the disinfection and the permanent filling of the root canals. The filling materials according to the present disclosure may have disinfecting properties lasting for at least 30 days.

The present disclosure concerns, in its broadest scope, a dental or bone filling material comprising liposomes and having disinfecting power with respect to the bacteria responsible for dental or bone infections, the liposomes being either liposomes charged with a bactericidal agent or another active molecule, or unladen cationic liposomes.

“Disinfecting power” in the sense of the present disclosure means the ability of a substance to inhibit or kill undesirable micro-organisms by altering their structure or their metabolism, regardless of their physiological state, in order to reduce their numbers. Thus, the filling materials according to the present disclosure have, in particular, a disinfecting power with respect to the bacteria responsible for dental infections, and may be sufficient to eliminate them, and, in particular, those that are resistant to a high pH (pH=12) induced by the calcium hydroxide-based filling cements, and which are responsible for post-filling dental infections.

A first object of the present disclosure concerns a dental or bone filling material, which is disinfectant against the bacteria responsible for dental or bone infections, comprising liposomes wherein:
said liposomes are either liposomes containing a bactericidal agent or unladen cationic liposomes,
the concentration of bactericidal agent released is above the minimum inhibition concentration for the bacteria.

The Minimum Inhibition Concentration (MIC) is the lowest concentration of a compound that inhibits visible bacterial growth. The MIC depends on the targeted bacterium and the agent used to destroy it. This reference concentration is therefore calculated for each experiment by first establishing a standard curve defining the concentration of bactericidal agent/percentage of bacteria eliminated. The minimum bactericidal concentration (MBC) corresponds to the elimination of over 99.99% of the bacteria. An agent is generally considered bactericidal if its concentration is 3 or 4 times the MIC.

This test can be applied to planktonic bacteria or bacterial biofilm.

There are several methods for measuring the disinfectant properties of a filler, including the Direct Contact Test (DCT) and the agar diffusion test.

For the direct contact test method, the disinfectant power is evaluated by measuring the optical density of a solution containing bacteria (e.g., at 650 nm for E. faecalis). The lower the optical density, the less bacteria in the solution and the more disinfecting the substance is.

In the case of the agar diffusion method, the disinfecting power is evaluated by measuring the inhibition zone (in mm). The larger the inhibition zone, the higher the disinfecting power.

Thus, in two alternative embodiments of the present disclosure, the filling materials have a disinfecting power evaluated as follows:
either by measuring the bacterial concentration at 7 days using the direct contact test method, the bacterial concentration being less than 10⁵ CFU/mL, preferably less than 10⁴ CFU/mL, and most preferably less than 1000 CFU/mL. This test can be performed on planktonic bacteria or on bacterial biofilm.
or by measuring an inhibition zone using the agar diffusion method, 24 h to 48 h after inoculation of the bacteria and placement of the filling material, the inhibition zone obtained with the disinfectant filling material being greater than that obtained with the control.

In a particular embodiment, the inhibition zone can be larger than 9 mm, preferably larger than 12 mm.

Bacteria responsible for dental infections, especially found in the root canals, include Streptococcus, Actinomyces, Enterococcus and Propionibacterium strains, particularly Enterococcus faecalis, which is resistant to pH values above 11.5. Up to 12 different species can be found in these canals.

Bacteria responsible for post-filling bone infections include staphylococci such as Staphylococcus aureus or coagulase-negative staphylococci, Propionibacterium acnes, etc.

The term “filling material” or “cement” or “filler cement” within the meaning of the present disclosure means any type of filling material, suitable for temporarily or permanently occluding a tooth canal or pulp chamber or a tooth cavity, or a bone deficiency.

The most commonly used dental filling materials are based on calcium hydroxide or calcium silicate, but any cement based on calcium disilicate or trisilicate, hydroxyapatite, silicone, zinc oxide-eugenol, resin, alginate and/or collagen may also be used. In particular, commercial cements having no disinfecting power may be used to prepare the filling materials according to the present disclosure. Examples include the commercially-available cements Sealapex™ and Apexit™, BIO ROOT™, RCS™, PRO ROOT MTA™, Pulp canal Sealer™, AH 26™, IROOT™ SP, TOTAL FILL™.

Bone filling materials are generally bioactive ceramics that can be natural or synthetic (coralline, Hydroxyapatite (HA), tricalcium phosphate sulphate (TCP), bioactive glass, calcium carbonate and calcium silicate). Chemically similar to bone, this class of cements shows high compressive strength and low ductility, which gives them a high resistance to deformation but also brittleness¹. Phosphocalcic ceramics are biocompatible, they have no osteogenic activity but are osteoconductive, providing support for bone cells and proteins. Interestingly, the contact of the matrix with the bone leads to the formation of osteoid tissue that gradually acquires the mechanical properties of bone. Among the most widely used, what phosphocalcic ceramics were made of was found:
Hydroxyapatite is very close chemically to the apatite of bone. It is considered to be osteoconductive but is not very absorbable².
Tri-calcium phosphate (TCP) of formula Ca₃(PO₄)₂ is found in an alpha or beta crystalline form. The most commonly used is beta-TCP. It is a porous ceramic that transforms into hydroxyapatite in vivo³. TCP is osteoconductive and undergoes rapid resorption by solubilization of the Ca and P ions used in new bone formation.
Bi-phase ceramics (BCP) combine HA and ß-TCP. This material combines the physical properties of each of the compounds: long-term stability for cell adhesion and absorbability allowing the creation of new bone tissue. Thus the HA/TCP ratio can be modulated according to the resorption rate and bone type.
Calcium phosphate or calcium silicate cements for injection. These cements come in the form of a paste obtained by mixing a powder (HA, Beta TCP, dicalcium phosphate dihydrate) and an aqueous solution. The solidification of the mixture occurs after the formation of a carbonate apatite of low crystallinity similar to bone.

In a particular embodiment of the present disclosure where the filling material can be or is a resinous cement, it is different from PMMA.

Filling materials can also be distinguished according to their use:

1) Use in dentistry for surface filling of dentine or for making a pulp dressing to replace dentine and pulp (capping).

2) Endodontic use in case of tooth devitalization to fill the pulp and the canal.

3) Use in bone surgery to fill a bone cavity after curettage.

A particularly simple temporary dental filling material can consist solely of calcium hydroxide and water or be prepared on the basis of these two components.

In the context of the present disclosure, the filling material is used as a matrix (or carrier) to which the liposomes are added. Liposomes are therefore not adsorbed on the surface of the material, but are mixed with the matrix of the material.

In a preferred embodiment, the filling material used is based on minerals, particularly calcium hydroxide, or on bioceramic-based minerals. In an even more preferred embodiment, the material is based on calcium silicate, in particular, tricalcium silicate (Bio Roof type) or calcium hydroxide, in particular, calcium dihydroxide (Sealapex type).

The liposomes associated with the filling material may be of a different nature. Their properties, alone or in combination with active compounds of the bactericidal agent type or any other active molecule, confer a disinfecting power to the filling material according to the present disclosure.

Particularly suitable liposomes are composed of cationic, neutral or anionic lipids comprising charged chemical entities selected from:

ALN-TEG-Chol: 8-(cholest-5-en-3β-xyloxy)-3,6-dioxaoctanyl alendronate,

CHEMS: Cholesterol hemi succinate,

Chol: Cholesterol,

Con-A: Concanavalin A,

DC-Chol: 3-ß[N1N1-dimethylaminoethane-carbamoyl) cholesterol,

DCP: Dicetyl phosphate,

DDAB: Dimethyldioctadecylammoniumbromide,

DMPC: 1,2-dimyristoyl-sn-glycero-3-phosphocholine, DMPG: 1,2-dimyristoyl-sn-glycero-

3-phospho-(1′-rac-glycerol),

DOPC: 1,2-dioleoyl-sn-glycero-3-phosphocholine,

DOPE: 1,2-dioleoyl-sn-glycero-3-phosphoethanolamine,

DODAB: (dimethyldioctadecylammonium bromide),

DOTAP: 1,2-dioleoyl-3-trimethylammonium propane,

DPPA: 1,2-dipalmitoyl-sn-glycero-3-phosphate,

DPPC: 1,2-dipalmitoyl-sn-glycero-3-phosphocholine,

DPPG: 1,2-dipalmitoyl-sn-glycero-3-phospho-(1′-rac-glycerol),

DPTAP: Dipalmitoyl trimethylammoniumpropane,

DSPC: 1,2-distearoyl-sn-glycero-3-phosphocholine,

DSPE: 1,2-distearoyl-sn-glycero-3-phosphoethanolamine,

PC: Phosphatidylcholine,

PEG: Poly(ethyleneglycol),

PI: Phosphatidylinositol,

PS: Phytosphingosine,

SA: Stearylamine,

EPC: 1,2-dioleoyl-sn-glycero-3-ethylphosphocholine,

DODAc: 1,2-dimyristoyloxypropyl-3-dimethyl-hydroxyethyl ammonium,

DMRIE: 2,3-dioleoyloxy-N-(2(sperminecarboxamide)ethyl)-N,N-dimethyi-1-

propananninium DOSPA: dioctadecylamidoglycylspermine

DOGS: 1,2-dimethyl-dioctadecylammonium bromide,

DDAB: 2-dioleyl-3-N,N,N-trimethylaminopropane chloride,

DOTMA: 1,2-dimyristoyl-3-trimethylammonium propane,

DMTAP: 1,2-distearoyl-3-trimethylammonium propane,

DSTAP: 1,2-dioleoyl-3-dimethylammonium propane,

phospatidylcholine,

phosphatidylethanolamine,

sphingomyelin,

phosphatidic acid,

phospatidylglycerol,

phospatidylserine,

phospatidylinositol,

DOBAQ: N-(4-carboxybenzyl)-N,N-dimethyl-1-2,3-bis(oleoyloxy)propan-1-aminium,

phospholipon 90G,

phytosphingosine.

Preferably, liposomes include DOPE, DOTAP, a mixture of DPPC: SA or DPPC:cholesterol, etc. DOPE is advantageously associated with HSPC.

Even more preferably, liposomes include DPPC and cholesterol.

After inclusion into the base material, liposomes can account for up to 20% (by weight) of the filling material, beyond that they destabilize and weaken the structure. Liposomes can represent, for example, 15%, 10%, 8%, 5%, 3%, 2% 1%, 0.5%, 0.1%, 0.05% or 0.01% of the material. In a preferred embodiment, liposomes represent between 0.01% and 15%, even more preferably between 0.5% and 10%, and most preferably between 1% and 5%.

Liposomes allow a better penetration of the bacterial biofilm [10; 11] and therefore a higher antibacterial efficacy. Indeed, they can fuse with the walls of the biofilm and thus deliver the active molecules to the heart of the biofilm, in addition to mechanically destabilizing the bacterial membranes.

In a first embodiment, liposomes are laden with a bactericidal agent or other active molecule. These liposomes can be cationic, neutral or anionic. They are loaded directly or indirectly with a bactericidal agent and, if necessary, another active molecule. Liposomes can be loaded after their formation (active loading), or during their formation (passive loading).

“Bactericidal agent” means at least one bactericidal agent, but may also be a combination of several bactericidal agents. The bactericidal agent may be selected from all local antiseptics and/or antibiotics that have demonstrated bactericidal activity. In particular, it can be chosen from the compounds listed in the ATC D08 class of the classification established by the WTO, which corresponds to “antiseptics and disinfectants”. Antiseptics suitable for dental use include copper oxide, zinc oxide, silver, chlorhexidine, triclosan, PVP iodine, QPEI, chitosan, nisin, NaOCl, cationic polymers, fluoride and bromide . . . . In a preferred embodiment, the antiseptic is chlorhexidine.

The term “bactericidal agent” in the meaning of the present disclosure covers the notions of “bactericidal agent” and “bacteriostatic agent”. Indeed, depending on the concentration of the agent, it will have a bacteriostatic action at low concentrations and a bactericidal action at high concentrations. These notions also depend on the intrinsic properties of the agent under consideration.

For the purposes of the present disclosure, “other active molecules” means any molecule having complementary activity to the bactericidal agent as such. This may be an antibiotic, for example, from the penicillin family, such as amoxicillin, or an antibiotic/antiseptic combination, such as nisin/tetracycline.

The encapsulation of bactericidal agents or other active molecules in liposomes allows protection of the latter against inhibition caused by biological fluids, the formation of precipitates or molecular complexes with the filling material, but also controlled and effective release over time.

In a particular embodiment, liposomes represent up to 10% (by weight) of the filling material (after incorporation of the liposomes) and these liposomes are loaded with bactericidal agent by hydration of a lipid film with a solution having a concentration of between 0.01% and 10%, preferably between 0.05% and 8%, even more preferably between 0.5% and 5%, most preferably between 1% and 5%. These percentages are before evaporation (knowing that the theoretical concentration is up to 4 times higher after evaporation) and are to be adapted according to the intrinsic disinfectant properties of the agents used.

The concentration of bactericidal agent encapsulated in the liposomes must be sufficient to release more than the MIC.

Liposomes loaded with a bactericidal agent can be cationic, neutral or anionic.

In another preferred embodiment, the liposomes loaded with bactericidal agent are neutral or anionic. These liposomes are neutral, and most preferentially they contain DOPE (e.g., DOPE: HPSC) or DPPC (e.g., DPPC:cholesterol).

In a particularly preferred embodiment, the liposomes are neutral and the active molecule is an antiseptic such as chlorhexidine.

In another preferred embodiment, the liposomes are a combination of liposomes of different natures (cationic, neutral, anionic) and the active molecules are an antibiotic and/or an antiseptic. An example of a different type of liposome association may be a mixture of DPPC: cholesterol: SA or HSPC: DOPE.

In another particular embodiment, the filling material comprises a cement based on calcium hydroxide or calcium silicate, liposomes consisting of neutral lipids and an antiseptic agent being chlorhexidine. The base cement is calcium trisilicate cement, the liposomes include DOPE or DPPC and the antiseptic agent is chlorhexidine.

Thus, the different embodiments can be combined to define a filling material with disinfectant power, as presented in the experimental section below.

A highly preferred embodiment is a calcium trisilicate-based filling material comprising 1% (by weight) of liposomes of the DPPC:chlorhexidine-loaded cholesterol type with a 1% chlorhexidine solution.

In a second embodiment, the liposomes are cationic and are not loaded.

When cationic liposomes are not loaded with bactericidal agents and, if necessary, active molecules, their disinfecting power is based on their ability to fuse with biofilms. By merging with the walls of the biofilm and the bacterial membranes, they disorganize the biofilm and the bacterial membranes leading to the destruction of the bacteria.

When the filling material comprises unloaded liposomes, these represent up to 20% (by weight) of the filling material, preferably between 0.01% and 10%, even more preferably between 0.5 and 10% and most preferably between 1% and 5%.

Regardless of the embodiment considered, the addition of liposomes in filling materials is particularly interesting for bacterial disinfection in endodontics because liposomes have an affinity for bacteria and can diffuse into the root canal network, dentin tubuli and periapex. Liposomes allow a more controlled diffusion of the antiseptic or batericidal agent than if this agent was alone in the filling material.

In all cases, whether the liposomes are loaded or not, their size must be small enough to allow them to penetrate the canals and tubuli. Tubule sizes generally range from 100 nm to 3200 nm, with an average of 800-1200 nm.

Thus, the “diameter” or “size” of the liposomes used in the present disclosure may vary between 20 nm and 8000 nm. Liposomes with such a diameter allow for an effective disinfection. However, it is necessary that the size is homogeneous and stable to ensure reproducibility of the results. The person skilled in the art will be able to define these adaptations.

In a particular embodiment, their size is less than 6000 nm, preferably between 600 nm and 50 nm, or even between 400 nm and 50 nm, more preferably less than 300 nm and most preferably between 100 nm and 200 nm, e.g., 150 nm.

The properties of disinfectant filling materials vary according to the chosen combination: nature of the cement, nature and concentration of liposomes and nature and quantity of active molecules. In addition, the nature of the cement and the nature and size of the liposomes can influence the diffusion of the liposomes and thus the release of the active molecules outside of the filling material in contact with the tooth or bone. It is important to control these parameters to control the quantity of antiseptics or other active molecules released in fine.

In a preferred embodiment of the present disclosure, the filling material is based on calcium hydroxide or bioceramics, and the liposomes are of the “DOPE liposome”, “EPC liposome” or “DODAP liposome” type, as defined in the experimental section below (Example 4.2).

In another preferred embodiment, the dental or bone filling material also includes a molecule from the family of surfactants (anionic, cationic, zwitterionic or non-ionic) such as block copolymers.

Molecules of the surfactant family (especially copolymers with dispersing power) promote the homogeneous distribution of liposomes in the material. As an example, the surfactant may be selected from Pluronic block copolymers (PEO—PPO-PEO), polyethylene glycol (PEG), polylactic acid (PLA), poly(D,L-lactic-co-glycolic acid) (PLGA), polycaprolactone (PCL), poly(ß-hydroxybutyrate) (PHB) or a mixture/blend of these molecules (e.g., Pluronic-PLA melt). The choice of the surfactant depends on the antiseptic to be encapsulated, the nature of the liposomes and the cement used. A concentration of the surfactant can vary from 0.1% to 20% (by weight) of the liposome solution. Particularly preferred surfactants are the pluronics, selected from the pluronics L31, L61, F68, F127, L43, L44, L62, L64, P85, P84, P104, P123, L35, F38, L42, L63, P65, F68, L72, P75, F77, P87, F88, F98, P103, P105, F108, L122, P123, F127, etc.

The choice of the surfactant depends on the antiseptic to be encapsulated, the nature of the liposomes and the filling material used. A concentration of the surfactant can vary from 0.1% to 20% (by weight) of the liposome solution, preferably from 1% to 10%, and most preferably from 1% to 3%. The addition of such a substance makes it possible to make the link between the liposome phase and the basic filling material, which will allow a more stable formulation of the liposomes avoiding burst release, but also a better dispersion of the liposomes in the material by avoiding aggregation phenomena, which ultimately results in a better release of the encapsulated product (homogeneous in quantity and time).

Particularly preferred filling materials according to the present disclosure are the following:

Sealapex® type calcium hydroxide cement with DOPE type liposomes;

Apexit® type calcium hydroxide cement with EPC type liposomes; and

Calcium silicate cement of the Total Fill® type with liposomes of the DODAP type.

In a particularly preferred embodiment, the filling material consists of a calcium trisilicate-based cement comprising 1% (by weight) anionic liposome solution, liposomes loaded with a 1% (by weight) chlorhexidine solution and containing 2% (by weight) pluronic L31.

Other combinations are possible such as: DOPE liposome encapsulating triclosan+Apexit®;

PEG2000 liposome encapsulating QPEI+Sealapex®;

DODAP liposome+Well Root®+PEG;

DOPE liposome encapsulating metronidazole+Bio Root®+F127 pluronic;

DOPE liposome encapsulating PVP iodine+Totalfill®+P85 pluronic;

DPPC: SA:Chol liposome+Bio Root®+Pluronic L61;

DOPE liposome encapsulating chlorhexidine+Well Root®+PLGA;

DPPC: Chol liposome+AH plus+PHB-PEG copolymer;

DPPC: Chol liposome encapsulating Nisin+AH plus+Pluronic L31;

DOPE liposome encapsulating PVP iodine+Calcium phosphate cement (Genex®, Biograft)+Pluronic F77;

DPPC: Cholesterol liposome encapsulating amoxicillin+Calcium phosphate cement+PEG;

DOPE: HSPC liposome encapsulating chlorhexidine+ionomer glass cement+Pluronic P85;

DOPE: HSPC liposome encapsulating chlorhexidine+Hydroxyapatite (Bio-Oss®)+PEG-PHB-PEG;

DOPE: HSPC liposome encapsulating zinc oxide+Biodentin+Pluronic L61; and

Phospholipon 90G: Cholesterol liposome encapsulating chlorhexidine+Bio Root™+PEG-PLGA-PEG copolymer.

The filling materials according to the present disclosure may be in the form of a powder-liquid mixture, granules or a premixed gel or paste capable of solidifying upon contact with air.

A second object of the present disclosure relates to a process for the preparation of a dental cement as previously defined comprising:

a) the preparation of liposomes with a size between 8000 nm and 20 nm,

b) mixing the liposomes with a dental cement in a proportion of between 0.001% and 20%, preferably between 1% and 5% liposomes (by weight).

The filling material thus obtained can be for dental or bone use.

In a preferred embodiment, a surfactant is added to the liposome preparation before mixing with the cement. This avoids the phenomenon of formation of micelles and then allows mixing between a hydrophilic cement base and hydrophobic liposomes.

Surfactants suitable for this use include the pluronics L31, L61, F68, F127, L43, L44, L62, L64, P85, P84, P104, and/or P123. In a preferred embodiment, the surfactant is selected from pluronics L31 and L61.

The addition of surfactant promotes the homogeneous distribution of liposomes in the cement and allows for good reproducibility of product quality, and, in particular, the quality required for the specification of dental products to be achieved.

When the liposomes are loaded with active molecules (antiseptic or other), the process further comprises a step of inclusion (or loading) of these molecules into the liposomes before mixing the liposomes with the cement.

The percentage of liposomes included in the filling material can be between 0.001% and 20% by weight, more particularly less than 20%, typically between 1% and 10% or 0.5% and 10%, preferably between 1% and 5% (by weight). This percentage will preferably be between 0.5% and 2%, but may vary depending on the size of the liposomes and their bactericidal load. In any case, the disinfectant power of cement must be controlled.

The filling material can be in the form of a powder-liquid mixture, granules or a pre-mixed gel or paste.

In the case of the powder-liquid mixture version, the resulting liposome solution is freeze-dried and the resulting powder is mixed with the base cement powder or paste depending on the commercial presentation of the base filling material. Freeze-dried liposomes are hydrated by the liquid of the filling material or by air humidity. Freeze-drying the liposome solution provides greater stability over time, allowing the filling materials to be stored at room temperature.

A third object of the present disclosure relates to the use of a dental cement as previously defined to prevent post-filling infections of the teeth or bone, in particular, of the root canal, the periapex and/or the dentin tubuli or of a bone cavity, in particular, in the jaw area.

Filling materials as defined above can be used in dentistry for temporary or permanent filling depending on the care protocol followed by the dentist. Thanks to their disinfectant power, they prevent a deep infection of the dental canals, which is difficult to treat and requires a new therapeutic intervention.

A fourth object of the present disclosure concerns a method for evaluating the disinfecting power of a filling material comprising:

(i) arrange, in a two-compartment container,

(a) a solution containing bacterial strains, which may be responsible for dental infection in the lower compartment, and

(b) a layer of filling material whose disinfecting power in the upper compartment is to be tested,

the two compartments being separated by a membrane perforated by pores with a diameter between 8000 nm and 50 nm;

ii) incubate the container at 37° C. for a selected period of time;

iii) assess the presence of bacteria in the lower compartment.

This experiment can be carried out by preparing two-compartment containers separated by a membrane with pores of appropriate diameter. It is also possible to use commercial devices such as Transwell® sheets sold by the Corning® Company.

The pore diameter of the membrane is custom selected. To reproduce the diameter of the tubuli, it will be chosen between 50 and 8000 nm, preferably between 300 and 800 nm and more particularly between 50 nm and 400 nm.

The incubation time will generally be between 2 and 48 hours. The person skilled in the art will know how to adapt this time according to conventional microbiology protocols.

The evaluation of the presence of bacteria will be carried out by one of the microbiological techniques known in the art, for example, by measuring the optical density of the solution by spectrometry.

This test is particularly relevant for assessing the disinfecting power of a dental filling material since the nanopores in the membrane mimic the size of dental tubules. If the bacteria are eliminated, this suggests that the cement will be effective in disinfecting the tubuli.

Finally, this test can be used to measure the disinfecting power of the filling material either against planktonic bacteria or against a bacterial biofilm, depending on the bacteria that will be added to the solution placed in the lower compartment.

The same tests can be used to validate the disinfecting power of a bone filling material, adapting the type of bacteria to be tested, if necessary.

The present disclosure shall be better understood by reading the following examples, which are provided by way of illustration and shall in no way be regarded as limiting the scope of the present disclosure.

### EXPERIMENTS

### Example 1: Preparation of Cements Comprising Liposomes 1.1−Preparation of Liposomes

Liposomes have been formed from the different lipids of interest described above using different conventional techniques: sonication or hydration of a lipidic film [9].

In all cases, residual organic solvents were removed to recover a pure powder, the total of which was then re-suspended in a biologically compatible solvent (usually water) to generate liposomes. This was obtained using evaporation techniques, which is not necessary if the liposomal detergent is fed into the aqueous phase at an adequate minimum concentration. For this proof of concept, medium to high quality reagents were required (>97.0% purity). For the preparation of dental cements, this should be improved to a minimum of >99.0% purity (HPLC purification).

One of the protocols used was adapted from previously published protocols [1, 2, 9].

In the preparation of the liposomes, a ratio (weight/weight) of the desired combination of lipids was achieved by accurately weighing the given detergent lipid prior to mixing in a large round-bottomed flask. To this, 5 ml of organic solvent (chloroform, HPLC grade) were added and the suspension was vortexed until the lipids were completely dissolved. The same flask was attached to a rotary evaporation kit, connected to a vacuum pump rotating at one revolution per second. The system was maintained at a generally constant temperature of 60° C. in a water bath, this temperature being determined by the phase transition of the lipid mixture of interest.

During the removal of any organic solvent, sterilized MQ deionized water was added to give a concentration of 5 mg.mL⁻¹. The suspension was then left for another 30 minutes to allow the liposomes to form.

The suspension was then taken and extruded ten times under 8 bar nitrogen pressure through a Lipex type extruder. The mixture was then extruded ten times in a second run through 400 nm polycarbonate membranes and again ten times in a third run through 100 nm polycarbonate membranes to achieve greater homogeneity in the size range of the liposomes. Finally, the suspension was centrifuged at 100,000 g for one hour (at 4° C.) to obtain a liposome pellet and remove excess water in preparation for the loading.

In these experiments, liposomes were prepared from DOPE, EPC, DODAP and PEG. 1.2—Inclusion of bactericidal compounds

The bactericidal compound is loaded into the liposomes by active or passive routes, depending on the chemical properties of the detergent lipid and the chemical compound used. Previously published studies have examined the loading of liposomes with the antibiotic gentamicin sulfate to form bone cement, which evidenced an improved therapeutic index of the antibiotic, as well as greater accumulation at sites of interest [2]. A related study examined the loading of the antifungal amphotericin B into liposomes to generate bone cement for active treatment [3].

Particularly interesting antiseptics are copper oxide, chlorhexidine, triclosan, PVP iodine and nisin, each of which can be loaded according to previously published protocols. Those that have been charged by active means (usually those of a hydrophilic nature) have been added using an externally generated gradient, in particular, by changing the pH [4] or electrochemically [5, 6]. Passive loading of chemical compounds is a simpler process, and in this case the chosen chemical compounds have been dissolved in the solvent/organic phase if hydrophobic or dissolved in the aqueous phase if hydrophilic.

In all cases, the concentration of the liposome samples was considered at all stages to help maintain the mixture at levels that take into account the CMC (critical micellation concentration) value. When studying lipids, the CMC is the concentration above which the solution will begin to generate surface-active micelles. Generally, the CMC is determined by hydrophobic-hydrophilic disaggregation in the sample: the greater the hydrophobic-hydrophilic disaggregation, the lower the CMC. The chemical basis of the lipid molecule also affects the CMC through its segment lengths, whereby longer segments result in lower CMCs. All of these factors were considered for this study.

1.3—Determination of the Size and Homogeneity of the Liposomes

The laser diffraction technique was used to determine the average diameter of the loaded liposomes and to confirm that the liposomes are not damaged as their size is in the range of 100 to 150 nm. This analysis, shown in FIG. 2, is performed using a Beckman Coulter N4 Plus particle size analyzer under conditions corresponding to those previously published [2].

Once the liposomes loaded, a surfactant (2% w/w) depending on the nature of the liposomes chosen was added. Thus, different types of surfactants were used such as: Pluronics L31, L61, F68, F127, L43, L44, L62, L64, P85, P84, P104, P123, F127.

1.4—Mixing the Liposomes with the Cement and Analysis of the Resulting Product

Once the liposome nanoparticles were formed, they were incorporated into the selected cement, mixed and prepared for testing. These techniques follow, with the indicated adaptation, previously published works [2]. In these trials, the cements used are the commercial cements Sealapex® and Apexit®_{.}

After the preparation of the liposomes, the final cement is obtained by mixing the liposome solution with the cement to obtain a final liposome concentration of 3% (by weight).

Alternatively, the final cement can be obtained by incorporating freeze-dried liposomes into the endodontic cement powder.

Transmission electron microscopy was used to visualize the liposome/cement mixture, in order to better understand the dispersion of liposomes in the matrix. The technique can be carried out as follows, by preparing a 1:1 ratio of the liposome nanoparticles with 4% w/v aqueous uranyl acetate and incubating the mixture for 60 minutes. The liposomes were concentrated by centrifugation as above and resuspended in 10 ml of a given cement, or the same volume of water as the control. The suspension was further diluted to a final concentration of 10% v/v and 10 μL of this added to a Formvar carbon film supported on a 400 Mesh nickel grid (EM Systems Support Ltd). The substrate was air-dried and each undiluted cement was tested. The dry nickel grids were analysed by 80 kV transmission electron microscopy (Philips CM12 TEM) and the images recorded for analysis.

### Example 2: Study of the Release of the Molecules Loaded into the Liposomes

The experiments described herein relate to the detection of the release of molecules encapsulated in the liposomes and the evaluation of the antiseptic power of the endodontic cement obtained. The tests were carried out with cement mixtures containing at the final volume each 0%, 1% and 3% (by weight) of loaded liposomes.

2.1—Release of Nanoparticles

a. Simple Release

The endodontic cements used in these experiments are commercially available. These are Sealapex® and Apexit® cements. They were prepared according to the manufacturer's instructions.

For each endodontic cement, the final volume of each liposome nanoparticle formulation is 0%, 1% and 3%. Cement was applied to the bottom of a 96-well plate, in triplicate. After the cement had set completely, 1 mL of PBS (pH 7.2) was poured into each well, then withdrawn and replaced with fresh solution according to the following kinetics: 4 hours, 8 hours, 12 hours, 24 hours, 36 hours, 48 hours and then once every 24 hours for 15 days.

A first release study was carried out using cements comprising liposomes loaded with rhodamine, without the addition of surfactant. The results of this study are presented in Table 1 below: they show a release of rhodamine in relatively small quantities.

TABLE 1
Amount of chlorhexidine released by the cements

| | | | As a |
|---|---|---|---|
| | | | percentage |
| | | Final released | of the starting |
| | 72 h | dose | dose |
| PEG | | | |
| MTA | 0.0875484 | 0.1750968 | 3.50154% |
| Biodentin | 0.1342 | 0.2684 | 5.36800% |
| Sealapex | 0.04554 | 0.09108 | 1.82160% |
| Apexit | 0.02861 | 0.05722 | 1.14440% |
| Epc | | | |
| MTA | 0.0319 | 0.0638 | 1.27600% |
| Biodentin | 0.0302 | 0.0604 | 1.20800% |
| Sealapex | 0.1291 | 0.2582 | 5.16400% |
| Apexit | 0.1086 | 0.2172 | 4.34400% |
| DOPE | | | |
| MTA | 0.0214 | 0.0428 | 0.85600% |
| Biodentin | 0.08266 | 0.16532 | 3.30640% |
| Sealapex | 0.1347 | 0.2694 | 5.38800% |
| Apexit | 0.107012 | 0.214024 | 4.28048% |

These results led the inventors to improve cement preparation by adding a surfactant to the liposome solution before mixing with the cement. The cements used in the remainder of the study include a tensioactive type surfactant.

b. Release through a porous membrane

The cement was placed in the upper compartment of the Transwell® insert on the porous membrane (400 nm). After the cement had set, the lower compartment was filled with 3 different fluids: water, saline or artificial dental fluid (artificial cerebrospinal fluid).

The presence of liposomes was measured by spectrophotometry at 1 h, 4 h, 24 h, 48 h, 72 h, 2 weeks and 3 weeks.

The same experiment was carried out with E. faecalis strains placed in the lower compartment (see Transwell® insert test). Three strains of E. faecalis were used: one identified under the reference ATCC 29212 and two others, RW35 and RN44 (see below for the culture and identification method).

### Example 3: Evaluation of the Antibacterial Properties of Liposome-Containing Cements

3.1—Direct Contact Test

The antibacterial efficacy of the nanoparticles was tested against E. faecalis, a common and resistant intracanal pathogen. To better confirm the antibacterial effect of the nanoparticles, they were tested after immobilization. To define the mode of action, the effect was further evaluated in the bacterial suspension.

a. Preparation of Bacterial Suspension

E. faecalis was cultured overnight in 5 mL of brain-heart infusion broth (BHI, Difco, Detroit, Mich., USA) at 37° C. under aerobic conditions. The upper 4 ml were transferred to a new test tube and centrifuged for 10 min at 4165×g. The supernatant was removed and the bacteria were resuspended in 5 ml of PBS and agitated for 10 s. The optical density of the bacterial suspension is adjusted at 650 nm, which corresponds to a transmittance of 90 T (0.5 on the McFarland scale: 1.5 10⁸ CFU/mL.

b. Antibacterial Effect of Immobilized Liposomes

The antimicrobial surface effect of endodontic cement incorporating 0%, 1% or 3% (by weight) liposomes was then evaluated. A microtitre plate (flat-bottomed 96-well plate, Nunclon, Copenhagen, Denmark) was positioned vertically and the side walls of 8 wells were coated with similar amounts of the material (10 mg) tested according to the procedure described above.

10 μL of the bacterial suspension were added on the surface of each cement and in empty wells. After inoculation (at 37° C. and saturation humidity) for 2, 5, 20 and 60 minutes, 240 μL of PBS was added to each well. After stirring for 1 minute with a pipette, the resulting solution was transferred and diluted serially. Bacterial survival was measured by culturing an aliquot of 20 μL of the solutions transferred to Triptic soy agar plates after serial dilution by a factor of 10. After incubation for 24 hours at 37° C., the colonies on the plate were counted and the CFU/mL was calculated.

The presence of bacteria was measured by spectrophotometry (at 1 h, 4 h, 24 h, 48 h, 72 h, 2 weeks and 3 weeks).

3.2. Transwell® Insert Test

A membrane with 400 nm pores was used to evaluate the diffusion of liposomes through these nanopores; these nanopores simulate, due to their size, the dentin tubules.

In a 96-well plate, the upper compartment was filled with 10 mg of cement with or without liposomes, and the lower compartment was filled with a solution comprising the bacterial strains.

Three strains of E. faecalis were tested: ATCC 29212, RW35 and RN44.

The disinfecting power was measured in the same way as in the direct contact test.

3.3. Agar Diffusion Test

An additional test was carried out to demonstrate the disinfecting power of the liposome-containing endodontic cement and compare it to that of cements without liposomes, which have limited disinfecting power, particularly on dental biofilm.

The test was performed in a Petri dish filled with a 10 mL layer of sterilized Muller Hinton agar. 4 mm-diameter wells were formed in this agar layer to contain the endodontic cements made under the same conditions as above.

In this experiment, the antibacterial activity against strains of Pseudomonas aeruginosa, Enterococcus faecalis, Staphylococcus aureus and Escherichia coli was evaluated.

The bacterial strains were cultured at 37° C. for 24 h in MH agar medium to produce a turbidity of 0.5 on the McFarland scale, corresponding to a concentration of 10⁸ CFU/mL. A layer containing these bacteria was deposited to cover the freshly mixed cements. The plates were stored for 2 h at room temperature and incubated at 37° C. for 24 h. A follow-up was carried out at 4 h, 8 h, 12 h, 24 h, 36 h, 48 h and then once every 24 h for 15 days: the inhibition zone in mm was measured with a calliper (visual test).

3.4. Modified Direct Contact Test (Biofilm)

This test demonstrates the disinfecting power of an endodontic cement against a bacterial biofilm (the major form of bacteria in the root canal). The biofilm was formed on 4 mm×4 mm×1.5 mm (width×length×thickness) slices of bovine incisors using a low-speed diamond disc under irrigation.

Samples are soaked in 17% EDTA for 3 minutes to remove the dentin sludge and then sterilised at 121° C. for 20 min.

A standard strain of E. faecalis (ATCC 51299) was used for the formation of the bacterial biofilm. The cultivation of E. faecalis was carried out under the same conditions as above. The bovine tooth slices were placed in wells of a 24-well plate. 220 μL of inoculum were added to each well, which was then topped up with 1.8 mL of sterile BHI solution. The wells were cultured at 37° C. under 5% CO₂ for 14 days. The supernatant was completely changed every 48 hours with fresh sterile BHI solution.

Endodontic cements prepared under the above conditions were placed on the biofilm-covered sections of bovine teeth.

The tooth/endodontic cement samples were placed in a new 24-well plate and stored at 37° C. under 5% CO₂ for 2.7 and 14 days.

After these incubation periods the remaining biofilms were transferred to tubes containing 1 mL of saline solution. The tubes were sonicated for 30 seconds at 40 W to separate the biofilms.

The number of bacteria (CFU/mL) was measured as described above.

### Example 4: Evaluation of the Percentage of Release of Fluorescent Molecules Contained in Liposomes Mixed with Cement

4.1—Aim of the Experiment

The objective of the following experiment was to evaluate the percentage of release of a fluorescent molecule (rhodamine) incorporated into liposomes mixed with endodontic cement. In practice, it consisted in comparing i) the release obtained from 10 mg of cement comprising 3% liposomes (by weight) containing rhodamine, which corresponds to the addition by weight of 1% lipids to the cement, versus ii) the release obtained with a cement comprising no liposome but only rhodamine (fluorescence alone).

The base cements used in this experiment were Apexit®, Sealapex® and Bio Root® RCS commercial cements. Liposomes were prepared from the following lipids: EPC, DOPE, DODAP.

4.2—Preparation of Liposomes

The liposomes were prepared using the method described above. The experiment was carried out with the following compositions: “DOPE liposome” (DOPE, HSPC), “DODAP liposome” (DODAP, PC, Cholesterol), “EPC liposome” (EPC, HSPC) in the following respective proportions (5:2:3 molar ratio), (3:7 weight ratio), (35:65 weight ratio)

A rhodamine solution (0.04 nmol) is used to hydrate the lipid film and obtain liposomes containing fluorescent molecules (liposome loading).

4.3—Preparation of the Filling Material Comprising Rhodamine-Loaded Liposomes

After obtaining the liposomes, they were mixed with 2% w/w Pluronic L31.

The previously prepared fluorescent liposome solution was then incorporated into the different cements tested, i.e., 100 mg of a given cement was mixed with 1 mg of lipids. 10 mg of the lipid/cement mixture were then placed in the wells of a 96-well plate, in triplicate. To prepare the “liposome-free” control cement, the rhodamine solution was incorporated directly into the cements in the same proportions as those used for the liposome-containing cements (same amount of rhodamine contained in the cement). The resulting mixtures were placed in an incubator at 37° C. and saturated in humidity.

4.4—Analytical Methods Used

Kinetics of release study:

After the material set completely (approximately 4 hours), 1 mL of PBS (pH 7.2) was added to each well, then removed and replaced with a fresh supernatant solution according to the following kinetics: 4 h, 8 h, 12 h, 24 h, 36 h, 48 h then once every 24 hours for 15 days.

Liposome Size:

The size of the liposomes was measured according to the method described above. The size of the liposomes was also measured after release from the cement.

Fluorescence Assay:

The rhodamine solution was used as the standard for the fluorescence determination.

At the moment of performing the assay, the supernatants were collected from each well and respectively transferred to a well of another plate used for the assay.

The assay was carried out by spectrophotometry (BIOTEK reader plate).

4.5—Results

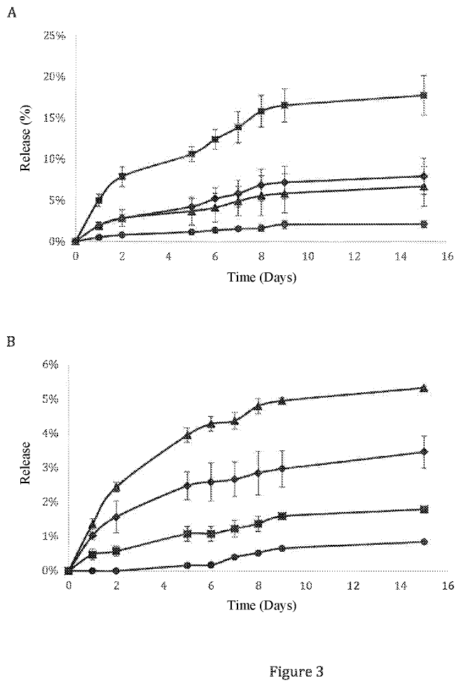

The results of this study are presented in FIGS. 2, 3 and 4.

Either liposomes encapsulating rhodamine or non-encapsulated rhodamine were incorporated into different types of cement.

Release of Rhodamine:

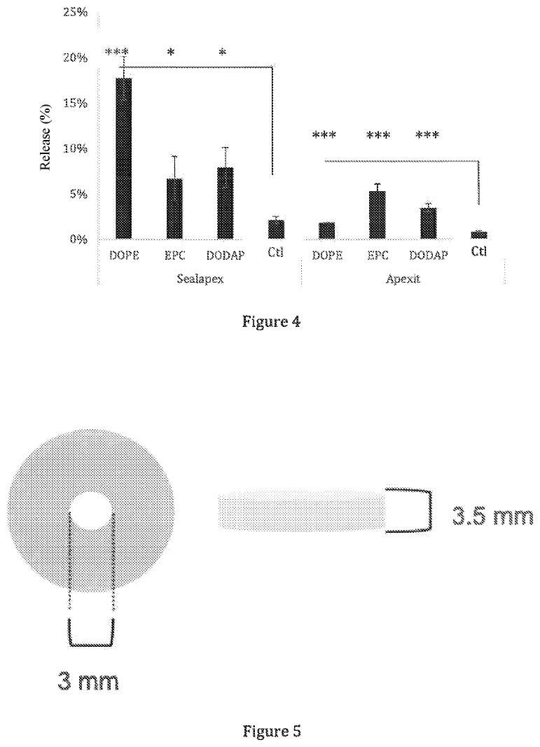

The results showed that liposome-containing cements allow a higher quantity and duration of release compared to cements that do not contain liposomes (the curves marked with a circle correspond to the cement without liposomes). This property is particularly interesting in endodontics, because it is necessary that the release be quantitatively sufficient and long-lasting to efficiently eliminate the bacteria [10; 11]. Consequently, these results show that the integration of liposomes allows the release of up to 19.43% of the amount of rhodamine incorporated in the cement as compared to only 1.67% when the rhodamine is not encapsulated in liposomes (FIG. 4).

Moreover, the total amount of rhodamine released in cements without liposomes occurs within 24-48 hours, after which time the cement releases very little fluorescent substance. The present innovation allows a diffuse release over a long period of 15 days. The developed cement makes it possible to achieve a minimum inhibition/bactericidal concentration that is constant over time, unlike endodontic cement without liposomes, which achieves this concentration for 24 hours (the minimum inhibition concentration cannot be achieved locally during the following days).

Finally, the results show that the combination of cement+liposomes encapsulating rhodamine allows a higher release than the combination of cement+rhodamine, whatever the type of cement and liposomes used. Nevertheless, the release profiles differ according to the lipids and the cements used.

Ultimately, the same release profiles as in previous studies associating bone cement and liposomes [1] were found. In addition, the formulation can be modulated according to the objective to be achieved, by modifying the nature of the lipids, the nature of the antiseptic and the nature of the endodontic cement.

In conclusion, liposomes were successfully incorporated into the filler cement to allow controlled release (in quantity and duration) of a molecule of interest. This innovation could play an important role in reducing post-treatment endodontic infections, which affect 44% to 77% of treated teeth. Improved disinfection through this innovation would allow fewer cardiovascular (infarction) and respiratory (sinusitis) complications and a reduction in the costs associated with the failure of endodontic treatment (antibiotics, crown, implant, etc).

### Example 5: Evaluation of the Release Percentage of Chlorhexidine Contained in Liposomes Mixed with Cement

5.1—Preparation of Liposomes Loaded with Chlorhexidine

Liposomes have been formed from the different lipids of interest described above using different classical techniques: sonication or hydration of a lipidic film or vortexing.

In all cases, the residual organic solvents were removed to recover a pure lipid film, which was then resuspended in a solvent (usually water) to generate liposomes. These were obtained by evaporation techniques, which is not necessary if the liposomal detergent is fed into the aqueous phase at a satisfactory minimum concentration. For this proof of concept, medium to high quality reagents were required (>97.0% purity).

In this trial, a different protocol from that used in previous trials was implemented. In this case, the lipids used were on the one hand DOPE and HSPC in a respective mass ratio of 30% and 70% and on the other hand DODAP, PC, cholesterol with a molar ratio of 5:2:3.

In the preparation of liposomes, the ratio (weight/weight or molar) within the desired combination of lipids was achieved by precise weighing of the given detergent lipid before mixing the lipids in a large round-bottomed flask. To this, 5 ml of organic solvent (chloroform, HPLC grade) were added and the suspension was vortexed until the lipids were completely dissolved. The same flask was attached to a rotary evaporation kit, augmented by a vacuum pump rotating at one revolution per second. The system was maintained at a generally constant temperature of 60° C. in a water bath, this temperature being determined by the phase transition of the lipid mixture of interest.

During the removal of any organic solvent, sterilized MQ deionized water containing 1% or 5% chlorhexidine digluconate was added to give a lipid concentration of 200 mg.mL⁻¹. The suspension was then mixed by vortex to allow the formation of liposomes with a size between 100 and 200 nm. At the end of this operation, 2% (weight/weight) of Pluronic L61 was added.

The liposome solution thus obtained can then be mixed with the endodontic or bone cement of interest. In this example, a 5% chlorhexidine solution with a liposome/cement weight ratio of 3% was used. The cement used was Sealapex.

The liposome solution was injected into the cement and manually mixed for 30 seconds.

Alternatively, the liposome solution obtained can be freeze-dried and the powder thus obtained can be mixed with the cement powder or paste depending on the commercial presentation of the cement. Freeze-dried liposomes are hydrated by the cement liquid or by air humidity. Freeze-drying the liposome solution provides greater stability over time, allowing the cement to be stored at room temperature.

5.2—Method for Calculating the Concentration of Released Chlorhexidine

The concentration of released chlorhexidine (CHX) was determined by spectrophotometry according to the following protocol:

a. Preparation of Cement Posts

The cement posts (filling material) were prepared in cylindrical Teflon molds with a diameter of 3 mm and a height of 3.5 mm (FIG. 5). The quantity of cement required to prepare 5 posts is 350 mg. The cement was weighed using an AB204 Mettler precision balance (Serial Nr P31828, last checked on 10/17/2016). For the Well Root™ and Total Fill cements, the 350 mg correspond to the commercial presentation. For Bio Root™, it consists of 250 mg of powder and 100 μL of liquid. A given quantity (see table Nr 3) of liposomes is added to the cement, which is then deposited using a cement spatula on a spatulation block (3M) for 30 seconds. The molds are then filled with a mouth spatula. The cement sets at room temperature for the Bio Root™ posts and at 37° C. in a humid environment (cell culture incubator) for the Well Root™ and Total Fill cements.

b. Measurement of Chlorhexidine Release

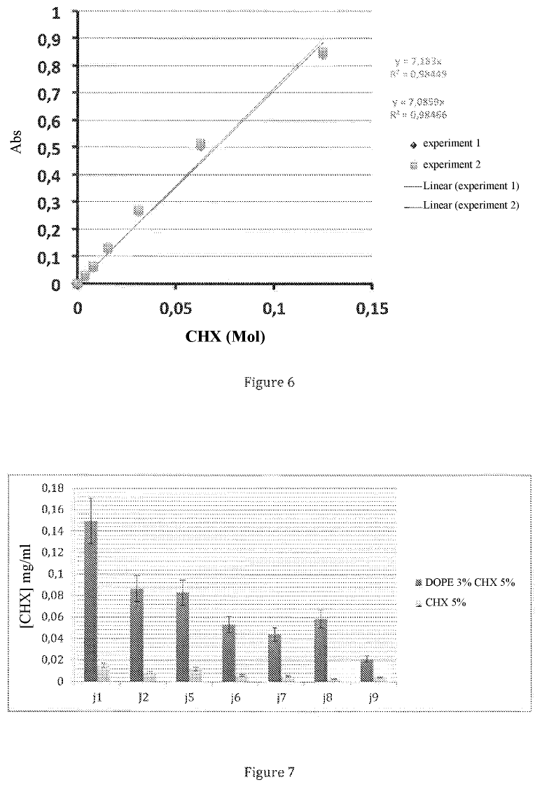

The posts are first weighed. In order to avoid maximizing the handling of the posts, they are placed in pre-weighed (tared) 1.5 mL Eppendorf tubes. The tubes containing the posts were then weighed to determine the weight of each post. 1 mL milliQ water is added to each tube. This volume corresponds to 10 times the volume of the post. The samples are then incubated at room temperature. Regularly, 100 microlitres of the solvent are removed and transferred to a well of a 96-well plate (Greiner Bio-One™ UV-Star™ 96-Well UV Spectroscopy Microplate). The absorbance is then measured at 255 nm using a UV/visible spectrophotometer (Xenius XLM, SAFAS, Monaco). This wavelength corresponds to the absorption peak of chlorhexidine diglucuronate (experimental verification before the start of the assay). 100 microlitres of milliQ water are then added to each sample to compensate for the volume taken for analysis. Absorption measurement at 255 nm was used to calculate the chlorhexidine (CHX) concentration of the solution from an experimental standard curve of chlorhexidine absorption versus concentration using the same spectrophotometer (FIG. 6: There is a linearity between the amount of CHX in solution and its concentration in the cement. This standard curve was used for the calculation of the released CHX concentration). The concentration is normalized by the weight of the posts and expressed as mg chlorhexidine/mL. The calculation of the concentrations takes into account the addition of 100 microlitres of milliQ water to replace the 100 microlitres of solution sampled for the measurement.

c. Chlorhexidine Release Measurement Results

The results are shown in FIG. 7. These show that the encapsulation of chlorhexidine in a liposome system and its injection into an endodontic cement such as Sealapex allows a significantly higher release of chlorhexidine than that measured from a cement containing chlorhexidine alone over a period of at least 9 days. In addition, the concentration of chlorhexidine released is higher than the MIC (concentration corresponding to the elimination of 90% of the bacteria) of Gram+ and Gram-bacteria; the thresholds being 0.022 and 0.04 mg/mL, respectively, (data from the literature).

### Example 6: Evaluation of the Disinfecting Efficacy of a Chlorhexidine-Loaded Liposome-Containing Filler Cement

6.1—Direct Contact Test

a. Test Conditions

This method was described previously in paragraph 3.1.

For this assay, Sealapex alone is used as the negative control (FREE Sealapex) and Sealapex mixed with chlorhexidine alone (without liposomes) (CHX Sealapex) as the positive control. Chlorhexidine alone is incorporated in the same proportions as that encapsulated in the liposomes.

b. Results

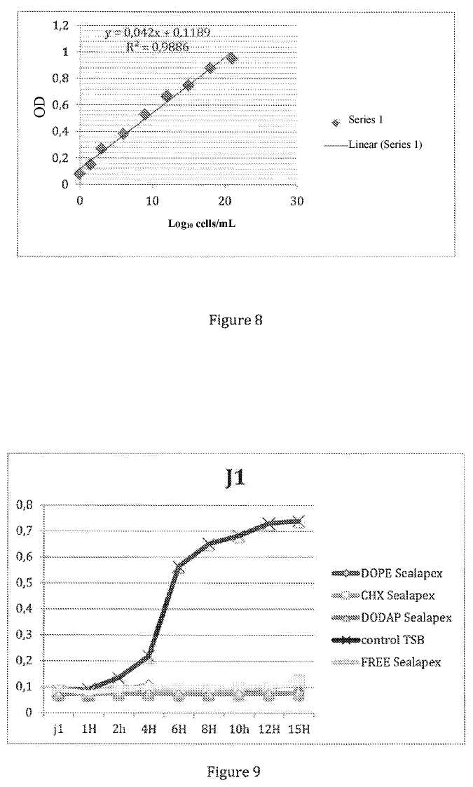

FIG. 8 shows the standard curve established for this test. This curve evidences a linearity between the concentration of bacteria and the optical density.

This standard curve was used for the calculation of the bacterial concentration and the measurement of the disinfecting power of the different formulations. Once the mixtures prepared at DO, the disinfecting power of the different cements was tested at D1, D7 and D20.

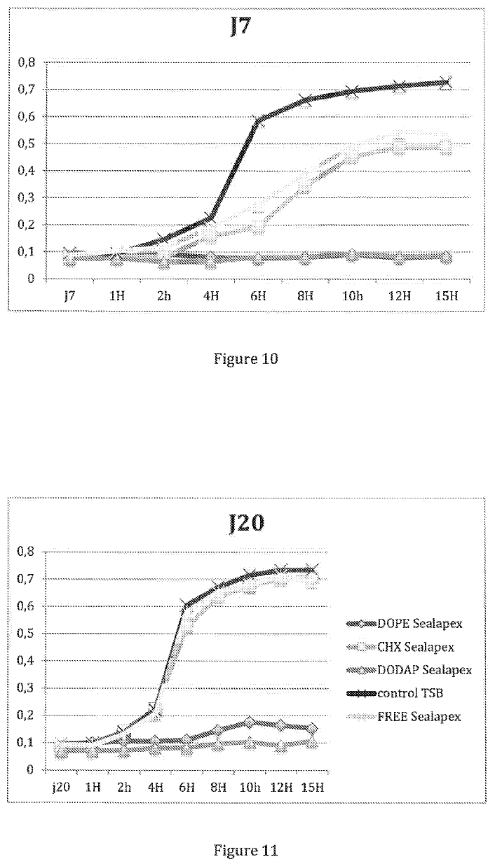

The results are shown in FIGS. 9, 10 and 11, corresponding to the results obtained at D1, D7 and D20,respectively.

It has been found that the integration of liposomes encapsulating chlorhexidine in a cement allows a higher disinfecting power to be obtained than that of a cement incorporating chlorhexidine alone and of the cement alone,up to at least 20 days. Also, 20 days after setting, cement alone or cement incorporating non-encapsulated chlorhexidine no longer has any disinfecting power, and bacteria are able to grow on it. The DOPE-Sealapex mixture shows a slight superiority over the DODAP-Sealapex mixture.

6.2—Modified Direct Contact Test

a. Conditions

This method was described previously in paragraph 3.4.

For this assay, the same cements as those tested in a. were used, the only difference in this case being that the bacterial culture medium (BHI) was used as negative control.

b. Results

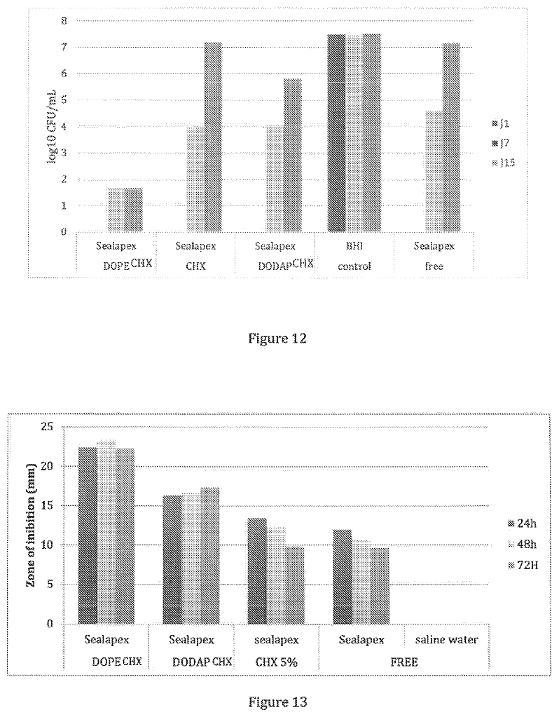

The results are shown in FIG. 12.

It is observed that the addition of liposomes encapsulating chlorhexidine eliminates 99.99% of the bacteria with an effect at least until D15 compared to an elimination of 54% of the bacteria for cement with non-encapsulated chlorhexidine. In addition, the liposome formulation of cement gives it bactericidal power, whereas cement without liposomes is bacteriostatic. The antibacterial superiority of the DOPE formulation compared to the DODAP formulation was found again.

Sealapex has a short term bactericidal action (D1) thanks to its high pH (12). Bacterial growth after D1 is due to a drop in pH and a selection of bacteria resistant to pH>12.

Tests were carried out with the same bacteria in the form of biofilm (14 days maturation). The results show the same trends, i.e., better disinfection of cement containing liposomes at D15 after contact (data not shown).

6.3—Agar Diffusion Test (ADT)

a. Conditions

This method is described in paragraph 3.3.

For this assay, the same cements as those tested in a. were used, the only difference in this case being that salt water was used as a negative control.

b. Results

The results are shown in FIG. 13.

The results show that liposome formulations with Sealapex achieve a larger zone of inhibition than the formulation without liposome at 24 h, 48 h and 72 h after incubation. These results confirm a higher disinfecting power of the liposome formulation of chlorhexidine against E. faecalis bacteria, mainly responsible for endodontic failures.

6.4—Conclusions

In conclusion, these tests show that cements containing liposome formulations containing a disinfectant molecule (in this case, chlorhexidine) and a surfactant (see description above) allow a diffuse and controlled release of the disinfectant molecule over a period of at least 15 days. This release is superior to that of a cement containing only chlorhexidine, which significantly improves the disinfecting properties of the selected cement.

It has also been shown that the addition of chlorhexidine alone improves the antibacterial properties of the cement little or not at all and that too much chlorhexidine addition leads to formation of a precipitate and alteration of the cement (disintegration). This phenomenon is due to the low release of chlorhexidine alone from the cement, and to the interaction of chlorhexidine with chemical elements in the cement such as phosphate groups.

The present disclosure makes it possible to reduce the bacterial load in an endodontic canal by effective diffusion of an antiseptic. This decrease would allow a reduction in the prescription of antibiotics and the number of endodontic treatment failures, which would lead to a reduction in prosthetic repairs and a decrease in edentulism, thus improving patients' quality of life.

Note that the Sealapex used in this test is a material with a hydrophilic and hydrophobic component. Many cements used today in dental surgery and orthopaedics are based on calcium silicate. As part of the development of the present disclosure, in a context close to the clinic, liposomes have also been integrated into calcium silicate-based materials in liquid or pre-mixed powder form (as described above).

### Example 7−Validation of New Filling Materials

With a view to the commercial development of new cements, new liposome production methods, new liposome formulations and new surfactants/polymers were tested.

The filling materials used are calcium silicate cements. Indeed, this type of cement is used for endodontic applications (filling material or endodontic cement), dental restoration (for example, Bio Dentine™), pulp capping/apexification (for example, Proroot MTA) or filling material in orthopaedics (for example, Cranioscult™). In addition, these materials can be used in combination with calcium phosphate.

For these trials, liposome formulations encapsulating chlorhexidine digluconate were prepared. The lipids DOPE, SA, Cholesterol and the CHX were supplied by the company Sigma Aldrich and the lipids DPPC and HSPC by the company Lipoid.

7.1—Process for the Preparation of Chlorhexidine-Containing Liposome Solutions

The following protocol was followed:

1. Solubilization of lipids in chloroform followed by evaporation in a rotary evaporator.

2. Hydration of the lipids with a solution of CHX (1 or 5%, 4 ml for 200 mg of lipids).

3. Extrusion of the liposomes (8 times) using a LIPOFAST LF-50 extruder (Avestin), with membranes with 100 nm pores.

4. Concentration of the formulations by an evaporator.

5. Measurements of DLS and Zeta potential.

6. Addition of 2% by weight of Pluronic L 31.

7.2—Produced Liposome Solutions

Following the protocol described above the following liposome formulations were produced:
DPPC: Chol: SA (cationic liposome), molar ratio 52.8:26:21.2, hydration in a 1% solution of CHX. Known as “DPPC 1%”
DPPC: Chol: SA (cationic liposome), molar ratio 52.8:26:21.2, hydration in a 5% CHX solution. Known as “DPPC 5%”
HSPC: DOPE (neutral liposome), weight ratio 7:3, hydration in a 1% solution of CHX. Known as “DOPE 1%”
HSPC: DOPE (neutral liposome), weight ratio 7:3, hydration in a 5% CHX solution. Known as “DOPE 5%”

Two types of liposomes were thus produced, associated with two different concentrations of CHX. The theoretical concentrations of lipids and CHX in the formulations were:

Hydration of 200 mg of lipids with 4 ml of a 1% CHX solution, then concentration to 1 ml.

200 mg/ml of lipids+40 mg/ml of CHX.

Hydration of 200 mg of lipids with 4 ml of a 5% CHX solution, then concentration to 1 ml

200 mg/ml of lipids+200 mg/ml of CHX

7.3—Liposome Size

The results are shown in Table 2 below:

TABLE 2
Size, zeta potential and poly dispersity of produced liposomes.

| | | | | | | Theoretical |
|---|---|---|---|---|---|---|
| Type of | | Theoretical lipid | Size | | Zeta | chlorhexidine |
| liposome | lipids | concentration | (nm) | Pdl | (mV) | concentration |
| DOPE 1% | DOPE:HSPC | 18% | 145.8 | 0.12 | 42.8 | 3.6% |
| DOPE 5% | DOPE:HSPC | 20% | 624.6 | 0.48 | 57.0 | 20% |
| DPPC 1% | DPPC:chol:SA | 20% | 5968 | 1 | 77.7 | 4% |
| DPPC 5% | DPPC:chol:SA | 20% | 927 | 1 | 81 | 20% |

Thus, it is observed that:
The size of the HSPC:DOPE liposomes hydrated with the 1% CHX solution did not change after concentration of the formulation (164.5 nm before and 167.7 nm after).
The size of the HSPC:DOPE liposomes hydrated with the 5% CHX solution increased after concentration of the formulation (106.1 nm before and 624.4 nm after).
The size of the DPPC:Chol:SA liposomes hydrated with the 1% CHX solution increased during storage (prior to concentration of the formulation). Note: The DPPC:Chol:SA liposomes hydrated with the 5% CHX solution were obtained by simple hydration.

7.4—Preparation of Filling Materials Containing Liposome Solutions

Filling materials were prepared by adding liposome solutions containing chlorhexidine to different types of calcium silicate cements. The samples were in the form of posts as shown in FIG. 5.

a. Protocol

For TOTAL FILL and WELL ROOT™ type cement fillers:

After manual mixing of the cement and the liposome solution, the mixture was vortexed for better homogenization and placed in plastic moulds until setting of the cement. As these two cements have a formulation that does not require a powder-liquid mixture, they are directly mixed with the liposome solution.
For BIO ROOT™ RCS cement-based filling materials: Since Bio Root™ is a liquid/powder mix, liposomes were added before mixing and the cement was prepared as recommended by the manufacturer (preferred method). Alternatively, the liposome solution can be added to the liquid in the case of Bio Root™ cement or added after mixing the powder with the liquid, and blended as described above.

For control endodontic cements, the chlorhexidine digluconate (CHX) solution is added in the same way and in the same proportions as the liposome solutions, to have the same percentage by weight of foreign matter in the cement and the same amount of CHX. After homogenization, the cement is considered ready and can be used for testing.

b. Description of the Obtained Filling Materials

The different combinations are summarized in Table 3 below

TABLE 3
Characteristics of the tested filling materials 7.5
- Study of chlorhexidine release a. Conditions

| | Total | Bio | Well |
|---|---|---|---|
| Cements tested: | Fill ™ | Root ™ | Root ™ |
| Mass of the sample: | 55 mg | 55 mg | 55 mg |
| Number of samples per combination: 6 | 6 | 6 | 6 |
| Number of combinations 24 | 18 | 18 | 18 |
| (16 lipidic + 8 controls) | | | |
| Liposomes tested: | DOPE | DOPE | DOPE |
| | DPPC | DPPC | DPPC |
| Lipid concentration in cement | 0.5% | 0.5% | 0.5% |
| | 1% | 1% | 1% |
| | 3% | 3% | 3% |
| | 10% | 10% | 10% |
| CHX concentration in liposome | 1% | 1% | 1% |
| | 5% | 5% | 5% |
| Concentration of CHX | N.A. | 1% | 1% |
| alone in solution | | 5% | 5% |
| Concentration of CHX | N.A. | 1% | 1% |
| alone in cement | | 10% | 10% |

The concentration of the released chlorhexidine (CHX) was determined by spectrophotometry according to the protocol described below:

The posts are first weighed. In order to better avoid handling the blocks, these were placed in pre-weighed (tared) 1.5 mL Eppendorf tubes. The tubes containing the posts were then weighed to determine the weight of each post. 1 mL milliQ water was added to each tube. This volume corresponds to 10 times the volume of the post. The samples are then incubated at room temperature. Regularly, 100 microlitres of the solvent are removed and transferred to a well of a 96-well plate (Greiner Bio-One™ UV-Star™ 96-Well UV Spectroscopy Microplate). The absorbance is then measured at 255 nm using a UV/visible spectrophotometer (Xenius XLM, SAFAS, Monaco). This wavelength corresponds to the absorption peak of chlorhexidine diglucuronate (experimental verification before the start of the assay). 100 microlitres of milliQ water are then added to each sample to compensate for the volume taken for analysis. Absorption measurement at 255 nm was used to calculate the chlorhexidine (CHX) concentration of the solution from an experimental standard curve of chlorhexidine absorption versus concentration using the same spectrophotometer (FIG. 6: There is a linearity between the amount of CHX in solution and its concentration in the cement. This standard curve was used for the calculation of the released CHX concentration). The concentration is normalized according to the weight of the blocks and expressed in mg chlorhexidine/mg cement. The calculation of the concentrations takes into account the addition of 100 microlitres of milliQ water to replace the 100 microlitres of solution used for the measurement

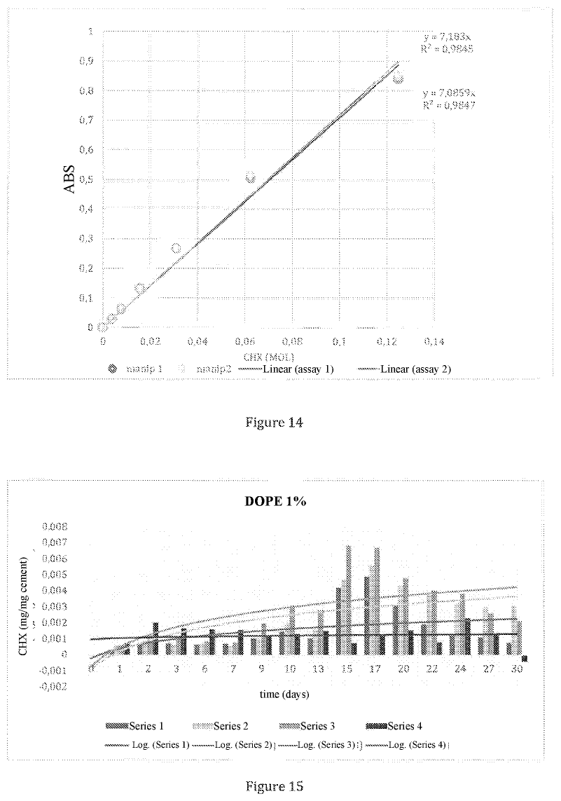

A new standard curve has been determined and is shown in FIG. 14.

A strong correlation was observed between the concentration of chlorhexidine and the absorbance of the solution at 255 nm. This curve was used to calculate the release of chlorhexidine.

Well Root™ and Bio Root™ RCS cements incorporating non-encapsulated chlorhexidine in the same proportions and theoretical CHX concentrations as in the liposome formulations were used as controls.

b. Results

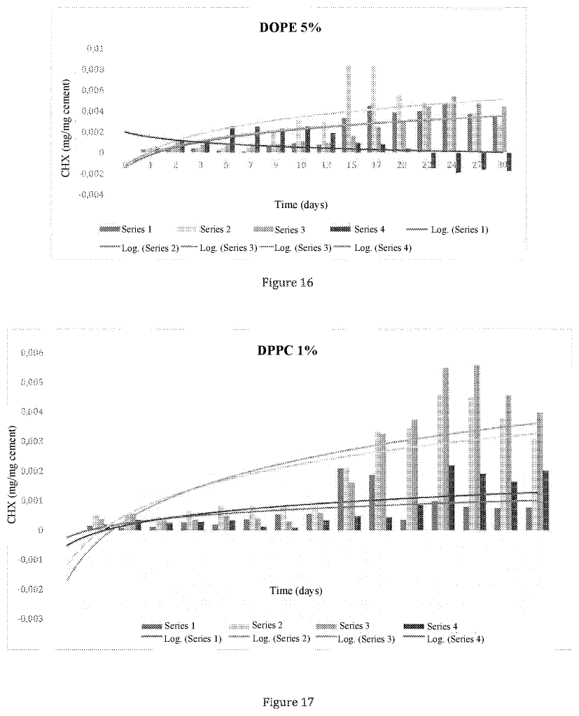

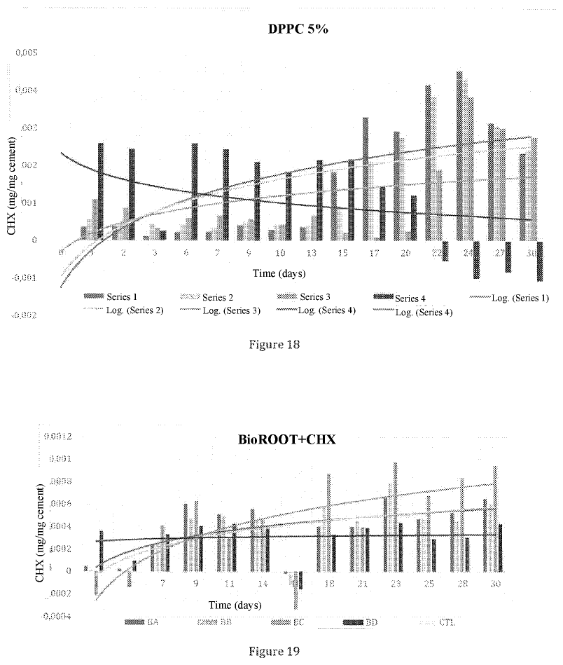

The results shown in FIGS. 15 to 19 were obtained with Bio Root™ base cement

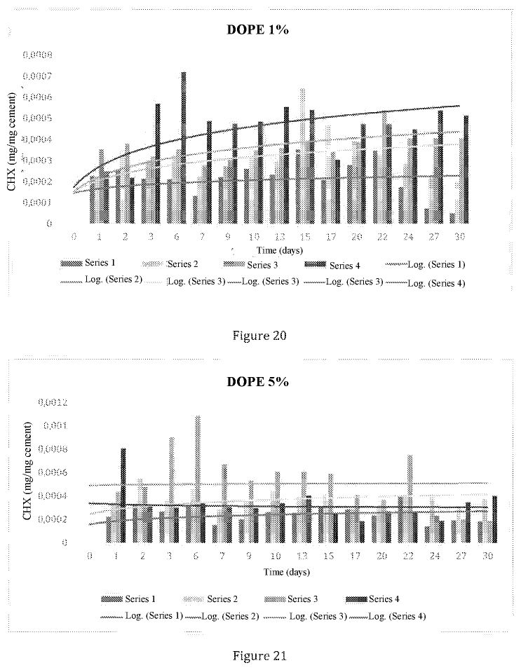

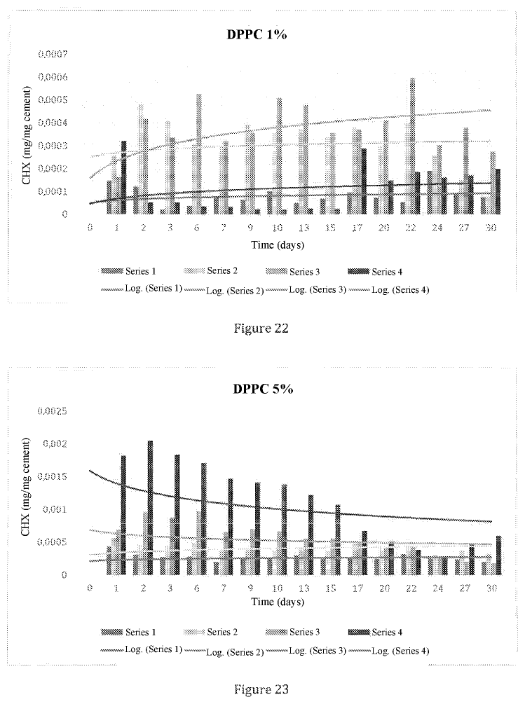

The results presented in FIGS. 20 to 24 were obtained with Well Root™ base cement
Results obtained with disinfectant Bio Root™ filler cement material

This combination showed different release profiles depending on the type of liposome and the theoretical chlorhexidine concentration.

It should be noted that the integration of at least 10% liposome alters the release profile regardless of the liposome used and regardless of the concentration of encapsulated chlorhexidine. The most reproducible concentration was that of a cement loaded with 1% liposome. The release is slow from D1 to D13 then accelerates between D13 and D20 to stabilize around D27. The maximum value achieved by all combinations is 0.008 mg CHX/mg Cement. It is observed that there is no direct proportionality between the concentration of encapsulated CHX and the CHX released. Also, too high a concentration of CHX seems to interfere with its release, even though the appearance of the released solution is clear. DOPE or DPPC cements with 1% encapsulated CHX release up to 90% of their theoretical CHX content. DOPE or DPPC cements with 5% encapsulated CHX release at most 30% of their theoretical CHX content. It should also be noted that the size and nature of the liposomes have less influence than the concentration of encapsulated chlorhexidine.

Lastly, the spatulation of the cement including the liposome solution and the setting time of the cement were the same as for cements without added liposome solution.

In conclusion, these results show that the integration of a liposome solution into a non-pre-mixed calcium silicate cement (Bio Root™ RCS) allows for an efficient release of chlorhexidine over a 30-day period. This integration can be performed at a weight ratio between 0.01% and 20% with regard to the cement, with encapsulated chlorhexidine concentrations between 0.1% and 20%.

With Bio Root™ cement, the optimal combination a 1% chlorhexidine solution-containing liposome solution included in the cement at a concentration of 1% by weight of the cement.
Results obtained with disinfectant Well Root™ filler cement material

Calcium silicate cements generally contain a thickening agent such as PEG.

The release was found consistent and diffuse for the DOPE 1% CHX formulation. This formulation produces a burst release in the first days (D0 to D3) and then a stabilization of the release occurs between D15 and D20 depending on the lipid concentrations in the cement. This formulation allows the release of a maximum of 20% of the amount of CHX incorporated in the cements. The other formulations show a close or equivalent amount of CHX released (between 15 and 20% of the amount incorporated in the cement) but with a less controlled release profile, especially for the DPPC 5% formulation. The maximum average CHX concentration released (0.0005 mg CHX/mg cement) is about 10 times lower than the maximum average CHX concentration released by Bio Root™. This could be explained by the different chemical natures of the cements and their respective porosities.

Concerning the influence of liposomes, it has been noted that neutral liposomes such as DOPE seem to be better adapted than cationic liposomes (DPPC). The size of the liposomes also influences the release pattern. It appears that the liposome formulations with sub-micron sizes release CHX more effectively. This could be explained by the small pore size of Well Root™ relative to Bio Root™.

In conclusion, the inclusion of a liposome solution in a pre-mixed calcium silicate cement allows for a controlled release in the case of liposomes that are sub-micron in size and neutral. Also, too high a concentration of encapsulated CHX seems to disturb the release of this molecule by possible chemical interactions with the cement.

With Well Root™ cement, the optimal combination is a 1% chlorhexidine solution-containing liposome solution at a concentration of 1% by weight of the cement.

Tests carried out with Total Fill™ cement, which is, like Well Root™, a calcium silicate cement containing a thickening agent, led to results similar to those obtained with Well Root™.

Conclusion: These experiments evidence the fact that the quality of the disinfectant filling material according to the present disclosure depends on three parameters: the nature of the cement, the nature and concentration of the liposomes and the nature and concentration of the disinfectant molecule.

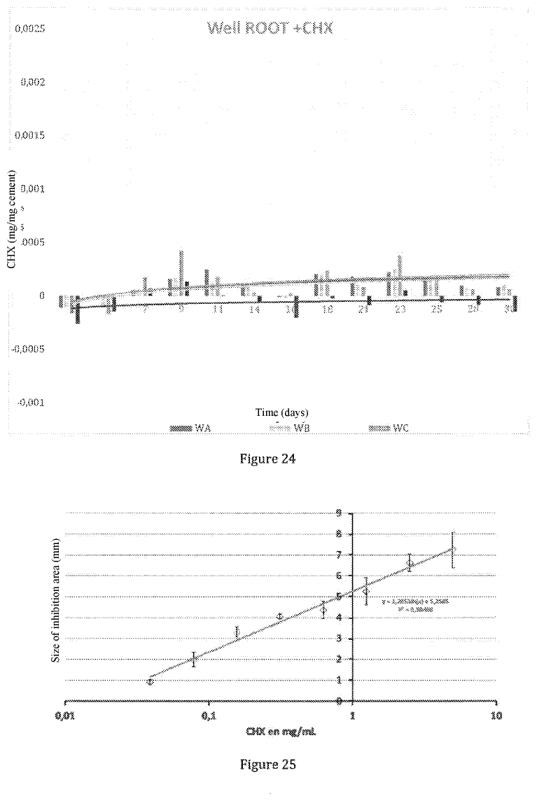

Calcium trisilicate cement of the Bio Root™ type without thickening agent gives better results than cements containing a thickener. The absence of thickener gives a purer and more porous cement that facilitates the release.
Comparison of the results obtained with cements containing only chlorhexidine (without any liposomes) The graphs of the results are shown in FIGS. 19 and 24.

It is observed that the inclusion of chlorhexidine within endodontic cements creates a white precipitate visible to the naked eye. This precipitate is probably generated by the reaction of chlorhexidine with phosphate groups present in mineral biomaterials. It should be noted that at a concentration above 5% of the CHX solution, the endodontic cement disintegrates completely and is not usable.

It is noted that the CHX released by these combinations is low and does not correspond to a controlled release. Most of the chlorhexidine release occurs during the first week in small or even nil amounts.

The average maximum concentration released by Well Root™ cement is 0.00015 mg CHX/mg for the best combination, as compared to 0.0005 CHX/mg cement in a liposome formulation of chlorhexidine.

Bio Root™ cement comprising only CHX releases an average maximum concentration of 0.0008 mg CHX/mg cement for the best combination, as compared to 0.005 mg CHX/mg cement for a liposome formulation; i.e., a 6.25 times higher release of the liposome formulation.

Furthermore, it should be noted that the release data for chlorhexidine alone have a much higher standard deviation (data not shown) than those with liposome solution, indicating a high variability, which is characteristic of uncontrolled release and low reproducibility (chemical reaction chlorhexidine-filler biomaterials).

In conclusion, the inclusion of chlorhexidine even at high concentration releases a low concentration that cannot be effective against bacteria.

The materials according to the present disclosure allow release, which is sustained over time, giving the filler biomaterial a better disinfecting power over a period of up to 25 days. Lastly, the encapsulation of an agent protects it against undesirable reactions: the inhibition of its action by some compounds, the formation of precipitates, the formation of harmful reaction products, etc.

This protection ultimately makes it possible to include a higher quantity of CHX, and therefore to obtain a high disinfecting efficiency, without damaging the physico-chemical properties of the biomaterial.

### Example 8—Study of Antimicrobial Activity of Disinfectant Filling Materials on Planktonic Solutions

8.1—Process for the Preparation of Liposomes and Filling Materials

The objective of these trials was to produce a liposome solution encapsulating chlorhexidine. The liposomes are composed of PHOSPHOLIPON 90G lipids and cholesterol in a molar ratio (2:1).

The oligolamellar liposomes encapsulating CHX were prepared by hydrating the lipid films at 30° C. with a 1% (by weight) aqueous solution of CHX. If necessary, mechanical agitation (vortex or sonication) was used to obtain complete hydration of the lipids. The resulting CHX-loaded liposomes were then extruded five times through two polycarbonate membranes with pores of 1 μm and 900 nm to obtain an average liposome size of less than 900 nm and homogeneity of the particles formed. The size distribution was determined by spectrophotometric techniques after extrusion. Pluronic L 31 was then added to the liposome formulation at a concentration of 0.5% (by weight) prior to concentration, after which the solution was concentrated to obtain a final lipid concentration of 200 mg/ml. The resulting solution is called CEM CHX 0918-001.

In order to verify that the concentration step had no impact on the structure of the liposome vesicles, the size of the concentrated liposome suspension was measured. At each point in time, the aspect of the formulation was examined before and after shaking the vials. The characteristics visible to the naked eye, i.e., turbidity, colour, aggregation, sedimentation and phase separation were analysed. The liposomes produced are then integrated—as described above—into the Bio Root™ RCS endodontic cement at a weight ratio of 1%.

Samples of disinfecting materials were produced in the same manner and under the same conditions as described in paragraph 5.2 a.

8.2—Agarose Gel Diffusion Test

a. Test Conditions

An adaptation of the agarose gel diffusion test was tested and validated for dental cements. The analysis was performed using the plaque assay. In the case of a test on samples of various concentrations, the ICM can be estimated using a software available on an on-line platform developed by Bonev in 2008 (http://agardiffusion.com) (Principles of assessing bacterial susceptibility to antibiotics using the agar diffusion method J. Antimic. Chemoth., 61 12951301).

For this experiment, liposomes and disinfecting fillers were produced as described in 8.1 and 7.6 a.

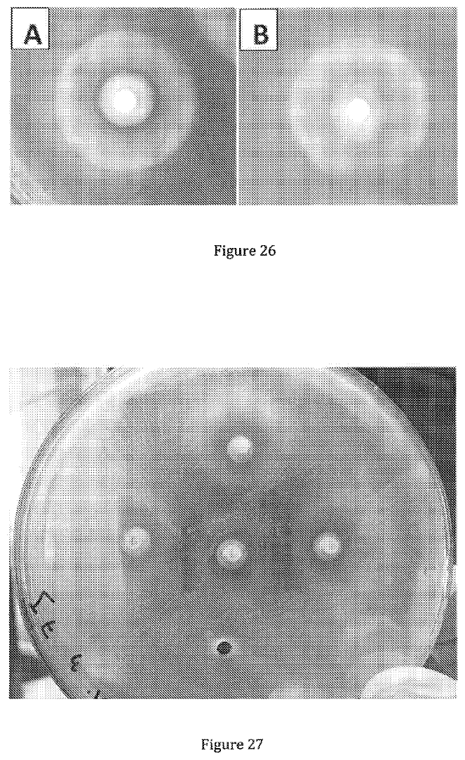

3 mm circular wells were punched using a disposable biopsy needle into Mueller-Hinton agar. The agar was then homogeneously inoculated with 100 μL of a solution of Enterococcus faecalis adjusted to a concentration of 0.5 MacFarland. Bio Root™ cement was then prepared (500 mg powder+250 μL liquid+/−35 μL CEM CHX 0918-001). The wells were then filled with cement using a pipetman for viscous solutions. The agar plates were then incubated at 37° C. under aerobic conditions for 24 hours. The area of growth inhibition was then measured using a ruler (FIG. 26).

To measure the effect of the prolonged release of antimicrobial agent, agar plates were prepared as above but without being inoculated with E. faecalis. They were kept for 7 and 14 days at room temperature. On the day of the test, a bacterial solution was prepared in BHI at a concentration of 0.5 MacFarland. 5 mL of the bacterial solution were added to the plates. These were then agitated for 30 seconds to obtain a homogeneous inoculation of the agar, then the medium was removed. The plates were then incubated for 24 hours and the lysis areas measured with a ruler.

b. Results

The calibration curve for this experiment is shown in FIG. 25. This curve represents the size of the inhibition area as a function of the concentration of chlorhexidine in the agar diffusion test.

The inhibition results at 24 hours (D1) are shown in FIG. 26. The inhibition results at D7 are shown in FIG. 27.

At D1 after the cement has set, there was no growth inhibition for Bio Root™ cement, whereas a growth inhibition area with an average diameter of 9.8±0.8 mm (n=9) was observed for Bio Root™ cement with 1% liposome solution added. The growth-inhibiting effect observed corresponds to a concentration of 40 μg/mL chlorhexidine according to a standard curve determined in a previous experiment with chlorhexidine digluconate (see FIG. 25). The calculated MIC of chlorhexidine digluconate for this experiment was 15.5 μg/mL.

At D7 there was no growth inhibition for Bio Root™ cement, whereas a growth inhibition area with an average diameter of 18±2 mm (n=8) was observed for Bio Root™ cement added with 1% CEM CHX 0918-001. It should be noted, however, that incubation for 7 days dries the agar and bacterial growth is less homogeneous. Nevertheless, the growth inhibition effect is undeniable (FIG. 27).

Conclusion:

The addition of 1% of CEM CHX 0918-001 confers an inhibitory activity on the growth of Enterococcus faecalis that is prolonged and amplified during the first 7 days. Amplification could be related to an extended release of the antimicrobial agent over time.

8.3—Antimicrobial Activity Test

The tests were carried out on two different bacterial strains: Enterococcus faecalis and Escherichia coli. These strains were incubated in planktonic culture in MHB (Mueller Hinton Broth) medium.

The growth was analysed in the presence of samples of filling material (cement) or in the presence of medium after incubation of the culture media for 24 hours at 37° C. under aerobic conditions. Growth was assessed by turbidimetry after 24 hours and 4 days of culture at 37° C. (according to the method described in Appl. Biochem. Biotechnol., 2014, 172:1652). Each measurement was carried out simultaneously on 3 samples and each experimental condition was tested 3 times (3 independent bacterial cultures), in order to have 9 measuring points for each condition.

Different sample volume to culture medium volume ratios were tested and a control with antibiotics (Tetracycline 10 μg.mL⁻¹+Cefotaxime 0.1 μg.mL⁻¹) was systematically included.

The aim of this experiment was to evidence the antibacterial activity of a product using a reference test.

8.4—Activity Test on Infected Dentin Slices

This test was intended to mimic the future conditions of use of the endodontic filling material, which must be in direct contact with infected dentine. Pre-sterilized human dentin slices 1 mm thick were incubated in a BHI solution inoculated with E. faecalis. The slices were incubated for 3 weeks to allow the bacteria to infect the dentin tubules.

The test therefore consisted in applying the filling material (cement) to one side of the infected dentin. The disinfected dentin slices were previously disinfected under conditions similar to endodontic treatment (6% NaCl for 5 minutes/17% EDTA for 3 minutes). When the cement had set, the tooth/cement assembly was incubated in BHI medium and re-cultured. A daily visual check determines when the medium is reinfected (turbidity control). About ten slices were for each type of cement. The entire surface was covered with cement. The result was expressed as the percentage of infected lamellae at time T. Fisher exact test was used to determine whether significant differences exist between the different conditions tested. In this case, only cements can be tested, but not the active ingredient alone.

Observing the lamellae by scanning electron microscopy allows contamination of the dentin tubules to be detected. Duration of the test: 6 weeks for each set of samples.

This test confirmed, under conditions similar to endodontic use, the disinfecting capacities of the filling material according to the present disclosure.

### REFERENCES

Ayre, N. W., Birchall, J. C., Evans, S. L. and Denyer, S. P. A novel liposomal drug delivery system for PMMA bone cements./Bionred Mater Res Part B. 2015; 00B:000-000.
Ayre, N W. Novel approaches to the development of PMMA bone cement. Thesis, PhD. 2013. Cardiff University.
Cunningham, B., McLaren, A. C., Pauken, C. and McLemore, R. Liposomal formulation increases local delivery of amphotericin from bone cement: a pilot study. Clinical Orthopaedics and Related Research. 2012; 470:2671-2676.
Mayer, L. D., Bally, M. B. and Cullis P. R. Uptake of 43driamycin into large unilamellar vesicles in response to a pH gradient. Biochemical Biophys Acta. 1986; 857:123-126.
Clerk, S. and Barenholz, Y. Loading of amphipathic weak acids into liposomes in response to transmembrane calcium acetate gradients. Biochemical Biophys Acta. 1995; 1240:257-265.
Haran, G., Cohen, R., Bar, L. K. and Barenholz, Y. Transmembrane ammonium sulfate gradients in liposomes produce efficient and stable entrapment of amphipathic weak bases. Biochemical Biophys Acta. 1993; 1151:201-215.
Ayre N. W., Birchall, L C, Evans, S. I., and Denyer, S. P. Liposomal Drug Delivery System for Bone Cements (WO 2015004450 A1). United Kingdom, PCT/GB2014/052085. 9 Jul. 2014.
Beyth, N., Domb, A. J. and Weiss, E. I. An in vitro quantitative antibacterial analysis of amalgam and composite resins. J Dent. 2007; 35:201-206.
Abolfazl Akbarzadeh, Rogaie Rezaei-Sadabady, [ . . . ], and Kazem Nejati-Koshki Liposome: classification, preparation, and applications
Katrien Forier, Koen Raemdonck, Stefaan C. De Smedt, Jo Demeester, Tom Coenye, Kevin Braeckmans
Lipid and polymer nanoparticles for drug delivery to bacterial biofilms
Kazuo Yamakami, Hideaki Tsumori, Yutaka Sakurai, Yoshitaka Shimizu,
Kohei Nagatoshi & Kenji Sonomoto
Sustainable inhibition efficacy of liposome-encapsulated nisin on insoluble glucan-biofilm synthesis by Streptococcus mutans
Ted J. Stowe, D D S, Christine M. Sedgley, BDS, MDSc, MDS, FRACDS, PhD, Bryant Stowe, and J. Christopher Fenno, PhD The Effects of Chlorhexidine Gluconate (0.12%) on the Antimicrobial Properties of Tooth-Colored ProRoot Mineral Trioxide Aggregate Journal of Endodontics, Volume 30, Issue 6, June 2004, Pages 429-431
N. B. Faria-Ju Nior, M. Tanomaru-Filho, F. L. C. V. Berbert & J. M. Guerreiro-Tanomaru, Antibiofilm activity, pH and solubility of endodontic sealers, Int Endod J. 2013 August; 46(8):755-62.
HARALD M ERIKSEN, LISE-LOTTE KIRKEVANG
Endodontic epidemiology and treatment outcome: general considerations;
¹ Gao et al, ‘Current Progress in Bioactive Ceramic Scaffolds for Bone Repair and Regeneration’.
² Daculsi, ‘Biphasic Calcium Phosphate Concept Applied to Artificial Bone, Implant Coating and Injectable Bone Substitute’.
³ Hollinger et al, “Role of Bone Substitutes.”

1.-14. (canceled)

15. A dental or bone filler material that is disinfectant against bacteria responsible for dental or bone infections, comprising:
a matrix material; and
liposomes, wherein the liposomes are selected from among liposomes carrying a bactericidal agent and unladen cationic liposomes.

16. The filler material of claim 15, wherein the matrix material comprises one or more matrix materials selected from among: calcium hydroxide, calcium silicate, calcium disilicate or trisilicate, hydroxyapatite, silicone, zinc oxide, eugenol, dental resin,alginate, collagen, hydroxyapatite, calcium phosphate, calcium carbonate tricalcium phosphate, fluoro-alumino-silicate, calcium sulphate, bioactive glass, and a two-phase ceramic.

17. The filler material of claim 15, wherein the liposomes have a size between 20 and 8000 nm.

18. The filler material of claim 15, wherein the liposomes comprise 20% (by weight) of the filler material.

19. The filler material of claim 15, further comprising at least one surfactant.

20. The filler material of claim 15, wherein the matrix material includes a cement including calcium hydroxide or calcium silicate, and wherein the liposomes comprise neutral lipids carrying a bactericidal agent.

21. The filler material of claim 20, wherein the cement includes calcium trisilicate, and wherein the liposomes comprise 1,2-dioleoyl-sn-glycero-3-phosphoethanolamine (DOPE) or 1,2-dipalmitoyl-sn-glycero-3-phosphocholine (DPPC) and carry chlorhexidine.

22. The filler material of claim 15, wherein the liposomes carry chlorhexidine.

23. The filler material of claim 15, wherein the liposomes carry an antibiotic, or a combination of an antibiotic and an antiseptic.

24. A method of forming a dental or bone filler material that is disinfectant against bacteria responsible for dental or bone infections, the method comprising the following steps:
a) preparing liposomes with a size between 20 nm and 8000 nm, the liposomes selected from among liposomes carrying a bactericidal agent and unladen cationic liposomes; and
b) mixing the liposomes with a dental cement in a proportion between 0.01% to 20% liposomes (by weight).

25. The method of claim 24, further comprising adding a surfactant to the liposomes prior to mixing the liposomes with the dental cement.

26. The method of claim 24, wherein preparing the liposomes comprises loading the liposomes with a bactericidal agent.

27. A method of filling a dental or bone cavity in a patient, comprising:
providing a filler material that is disinfectant against bacteria responsible for dental or bone infections, comprising:
a matrix material; and
liposomes, wherein the liposomes are selected from among liposomes carrying a bactericidal agent and unladen cationic liposomes; and
inserting the filler material into the dental or bone cavity in the patient.

28. A method for evaluating a disinfecting power of a filler material, comprising:
(i) arranging, in a two-compartment container,
a. a solution comprising bacterial strains, which may be responsible for a dental or bone infection in a lower compartment, and
b. a layer of the filler material in an upper compartment, the two compartments being separated by a membrane with pores having a diameter between 50 nm and 8000 nm;
(ii) incubating the container at 37° C. for a selected period of time; and
(iii) assessing a presence of bacteria.
